(19) United States
(12) Patent Application Publication
(10) Pub. No.: US 2023/0346829 A1
(43) Pub. Date: Nov. 2, 2023

(54) COMPOSITIONS AND METHODS FOR TREATING VIRAL INFECTIONS

(71) Applicant: ReVir, Inc., Buena Park, CA (US)

(72) Inventor: Joseph Lee, Buena Park, CA (US)

(73) Assignee: ReVir, Inc., Buena Park, CA (US)

(21) Appl. No.: 17/998,644

(22) PCT Filed: May 11, 2021
(86) PCT No.: PCT/US2021/031872

§ 371 Date: Nov. 11, 2022

Related U.S. Application Data
(60) Provisional application No. 63/166,214, filed on Mar. 25, 2021.
(60) Provisional application No. 63/133,175, filed on Dec. 31, 2020.
(60) Provisional application No. 63/023,124, filed on May 11, 2020.

(51) Int. Cl. A61K 31/739; A61P 31/12; A61K 31/573

(52) U.S. Cl. CPC A61K 31/739; A61P 31/12; A61K 31/573

(57) ABSTRACT

Provided are methods of treating a mammal for a virus infection. The methods comprise (a) increasing ribonuclease amount or activity in a cell infected by the virus; (b) decreasing protein production in a cell infected by the virus; (c) activating an innate immune system in the mammal; (d) reducing migration and/or activation of white blood cells in the mammal; or any combination thereof. Also provided are compositions effective in treating a virus infection when administered to a mammal having the virus infection.

### CROSS-REFERENCE TO RELATED APPLICATIONS

This application claims the benefit of U.S. Provisional Application No. 63/023,124, filed May 11, 2020, U.S. Provisional Application No. 63/133,175, filed Dec. 31, 2020, and U.S. Provisional Application No. 63/166,214, filed Mar. 25, 2021, all of which are incorporated by reference herein in their entirety.

### BACKGROUND OF THE INVENTION

### (1) Field of the Invention

The present application generally relates to relates generally to viral respiratory infections and includes materials and methods for the treatment for colds and viral infections including respiratory tract infections.

### (2) Description of the Related Art

A virus is the smallest microorganism with the ability to infect the cells of a living organism and beget illness or disease. Respiratory viral infections account for the most illnesses in the United States, and are the leading cause of disease worldwide. A viral respiratory infection, or VRI, refers to a virus that specifically affects the lower or upper respiratory tract, with the rhinovirus and influenza virus being the most prevalent. As the specific anatomical location of the targeted area within the respiratory tract varies with the specific pathogen, so do the particular viral syndrome that results and the severity of the illness that ensues.

Viral respiratory infections are usually clinically diagnosed based on symptoms and are generally classified according to syndrome. These clinical syndromes can be mapped anatomically along with the associated virus that is responsible. Generally divided into the upper and lower respiratory tract infections, the latter consists of a greater degree of severity of illness within its patients. Upper respiratory tract infections commonly include rhinitis, pharyngitis and laryngitis, while infections of the lower respiratory tract include croup, bronchitis, bronchiolitis, and pneumonia.

Infectious rhinitis, or the common cold, involves inflammation of the nasal cavity and the typical viruses responsible for causing rhinitis are the rhinovirus and the coronavirus. The rhinovirus accounts for roughly half of all common colds and it consists of over 100 different serotypes. The coronavirus, on the other hand, is a family of viruses, with only four serotypes being commonly responsible for these mild to moderate infections: 229E, OC43, NL63 and HUK1. Aside from these two groups of viruses, many others, including the enteroviruses, influenza viruses, parainfluenza viruses, adenoviruses, respiratory syncytial viruses, and metapneumoviruses can cause infections that may also manifest as the common cold.

Further down the upper respiratory tract are the nasopharynx, oropharynx, and larynx, and when infected, clinical syndromes of pharyngitis or laryngitis occur. Adenoviruses and parainfluenza viruses (type 2) typically cause these particular upper respiratory infections (URIs).

Lower respiratory tract infections occur below the vocal cords and are generally more severe, mainly due to their ability to affect gas exchange. Bronchioles, or the smaller airways, tend to more easily fill up with mucus, causing respiratory problems and sometimes requiring hospitalization. Laryngotracheobronchitis, or croup, is a classic, clinical syndrome that commonly manifests in children. It involves swelling of the area right below the vocal cords and is caused by the parainfluenza virus type 1.

Infections further down the lower respiratory tract include bronchitis, where a persistent cough develops in patients as a mechanism to clear the secretions caused by the inflammation of the mucus membranes of the bronchi. The agents that can cause this infection are varied, ranging from parainfluenza virus, influenza virus, adenovirus, metapneumovirus, RSV, and coronavirus.

The final major type of viral respiratory infection is pneumonia, a potentially serious and sometimes life-threatening complication for its hosts. It occurs at the ends of the bronchiole trees at the alveoli, where gas exchange takes place, and infection of the alveolar epithelium causes inflammation around the involved areas, decreasing gas exchange and resulting in serious consequences such as hypoxia. The influenza virus accounts for most cases of viral pneumonia, but this illness can be caused by all of the respiratory viruses. The influenza virus is classified as Type A, B, or C by its nucleoproteins and matrix proteins. Only a few combinations of these 18 H types and 11 NA types are human pathogens, but 6 major pandemics have occurred to date over the last 2 centuries, including the Swine influenza (H1N1) in 2009.

The coronavirus is one of the respiratory viruses that can cause respiratory failure, and one particular strain is currently garnering much notoriety for causing the illness that is COVID-19. Unlike the four strains associated with the common cold, the remaining 3 coronaviruses are much more severe and sometimes even fatal, accounting for major outbreaks of lethal pneumonia in the past century: SARS-CoV and MERS-CoV were first identified in 2002 and 2012 respectively, and COVID-19 spread globally from its origin in Wuhan, China in late 2019. The onset of COVID-19 stemmed from this zoonotic pathogen, which possesses formidable person-to person transmissibility. The dangerous nature of COVID-19 lies in that infected individuals may be asymptomatic after initial inoculation sometimes for days, and patients with immune-deficiencies, other underlying medical disorders, or simply old age, are at risk of falling severely ill and even succumbing to death. Symptoms can include fever, dry cough, difficulty breathing, and muscle pain, and various measures are being applied at global, national, and local levels to combat the contagious nature of COVID-19. Treatment of the disease is supportive, with no vaccine or antiviral drug yet available, although over 175 treatment and vaccine clinical trials are currently registered.

### BRIEF SUMMARY OF THE INVENTION

As described below, the present invention features compositions and methods for treating viral infections, particularly respiratory viral infections. The invention provides a method of treating a respiratory virus infection in a subject, the method involving providing a patient who has developed a respiratory virus infection with a factor (or agent) that induces production of interferon, thereby facilitating the signaling process the body uses in the normal course of healing from a virus infection while also providing an agent that decreases the recruitment and/or activation of white blood cells.

### BRIEF DESCRIPTION OF THE SEVERAL VIEWS OF THE DRAWINGS

The foregoing and other objects, aspects and advantages will be better understood from the following detailed description of the preferred embodiments of the invention with reference to the drawings.

FIG. 1 is a schematic illustrating the fundamental strategies within cells and between cells.

FIG. 2 is photographs and a drawing illustrating ImageJ image processing and particle analysis of cell cultures.

FIG. 2A shows a raw photographic image taken with Inverted Tissue Culture Microscope using a 4× objective lens.

FIG. 2B shows the raw image of FIG. 2A after processing has been completed. Processing includes converting the image to 8 bit black and white, adjusting the brightness/contrast and conversion to a binary sample.

FIG. 2C shows the resultant image after particle analysis. The image illustrates the outlines of all cells detected.

FIG. 3 is photographs showing cell culture images over time. This qualitatively illustrates cell culture growth over time. The top row represents the control culture, and the bottom row are the cells cultured with LPS and cortisol.

FIG. 4 is a graph of cell population increase over time. The bar graph quantitatively represents cell culture growth (number of cells) over time. The y-axis represents the number of cells detected by ImageJ particle analysis and the x-axis represents time in days. Error bars were set at a standard value of 5%, which is close to the average mean error percentage results when using ImageJ automated particle analysis program.

### DETAILED DESCRIPTION OF THE INVENTION

### Current Drug Search Strategies

Current drug searches for treatment of COVID-19 often focus on a very specific viral protein. The irony is that the drug, even if very specific for the COVID-19 virus or a viral protein, has to be taken orally and is often distributed widely throughout the body. Almost all lists of potential drugs against coronavirus are derived from a specific study of the genes of the coronavirus and involve an attempt to interfere with the virus proteins and their specific functions. It is classic for the authors to make a statement like, “New coronavirus protein reveals potential drug target”. Or, “scientists have identified a vulnerable site on a protein common to all coronaviruses”. Or, “researchers have found a new peptide that can bind to the coronavirus spike protein”. This approach does not take into account how the body actually rids itself of billions of coronavirus particles, often within days. True, even if the understanding of the precise mechanism whereby the body rids itself of the coronavirus is not correct or not understood correctly, this game of attempting to target specific viral proteins can still produce an effective therapeutic drug. However, imagine if there is a better understanding of how the body actually clears itself of the viral infection. Now, there are even more ways of potentially treating coronavirus. The focus is always on how to “block” some function of a coronavirus protein. Imagine a paradigm that correctly mechanistically explains how respiratory viral illness is overcome so easily in millions of patients by over 95 percent of the population. With a correct paradigm, the focus does not have to be to just “block” a coronavirus protein with a new drug, but a whole class of other potential therapeutic candidates now includes compounds that can “potentiate” a method the body is already actively using.

### Definitions

As used herein, the term “respiratory tract” includes the upper respiratory tract (e.g., nasal passages, nasal cavity, throat, pharynx), respiratory airways (e.g., larynx, trachea, bronchi, bronchioles) and lungs (e.g., respiratory bronchioles, alveolar ducts, alveolar sacs, alveoli).

The terms “nucleic acid” and “polynucleotide” as used herein refer to an oligonucleotide, polynucleotide or nucleotide and fragments or portions thereof, and to DNA or RNA of genomic or synthetic origin, which may be single- or double-stranded, and represent the sense or antisense strand. The term should also be understood to include, as equivalents, analogs of either RNA or DNA made from nucleotide analogs, and, as applicable to the embodiment being described.

An “effective amount” or a “sufficient amount” of a substance is that amount sufficient to cause a desired biological effect, such as beneficial results, including clinical results, and, as such, an “effective amount” depends upon the context in which it is being applied.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

### Abbreviations

TLR, Toll-like receptor;
PRR, pattern recognition receptor;
PAMP, pathogen-associated molecular pattern;
RLR, RIG-I-like receptor;
dsRNA, double-stranded RNA;
ssRNA, single-stranded RNA;
MyD88, myeloid differentiation primary response 88;
IRF, Interferon regulatory factor;
NF-κB, nuclear factor-kappa B;
IKK, IκB kinase;
PAMPs, pathogen-associated molecular patterns;
ISGs, IFN-stimulated genes;
OAS, 2′,5′-oligoadenylate synthetase;
IFNAR, type I interferon α/β receptor;
IKK-I, inducible IκB kinase;
IRF3, interferon regulatory factor-3;
ISGF3, IFN-stimulated gene factor-3;
NOD, nucleotide-binding oligomerization domain;
PKR, dsRNA-dependent protein kinase;

When challenging the status quo, a correct understanding of whether there is a problem with the current paradigm in any given area is a huge hurdle. Once the problem is properly presented with the current paradigm, it is easier to accept an alternate view.

The strength of a scientific theory is related to the diversity of phenomena it can explain and its simplicity. As additional evidence is accumulated, a theory may be modified or ultimately rejected if it cannot be made to fit the new findings; in such circumstances, a more accurate theory is then required. The current paradigm for healing and recovery of viral respiratory illnesses revolve around the IgG antibody as a central player in this fight against the virus. There are several situations that are not satisfactorily explained with the current paradigm. There are also some glaring gaps in terms of amount of time to generate the specific virus directed IgG antibody generation versus the amount of time for an average patient to completely recover from respiratory viral illnesses. A substantial antibody response may take 14 days. A patient may have completely recovered in 7 days. Just this fact alone should cause significant concern with the current paradigm which has the virus specific antibody as the main actor.

A neglected but very important question is, how a typical patient can recover from a mild flu or cold within a few days. It will become apparent that there is a major error with the current understanding of how respiratory viruses propagate and how the body actually eradicates a viral infection. For example, if a patient feels the symptoms of a mild cold or flu, at the minimum, many thousands of cells in the upper respiratory tract must be infected with the virus. If a virus is able to propagate within a host cell and multiply and then subsequently invade other neighboring cells, there is no obvious reason why the virus propagation cycle should stop, prior to an adequate antibody response. Clearly, many millions of antibodies are necessary to mount a successful defense against this virus. But it is highly unlikely that the body can mount a significant antibody response before 7 to 10 days. Yet, many people feel a mild cold or flu and then the symptoms resolve with little further sequelae in a few days. In these cases, clearly the antibody paradigm can't explain how the viral infection which had gained a foothold, then resolved. It is very notable that in approximately 95 to 99% of all COVID-19 cases in the U.S., infected patients recover before 14 days. It is extremely notable then, that the current paradigm using antibodies as the major player cannot explain how 95% of COVID-19 cases in the U.S actually recovered or healed. Prior to COVID-19, with only influenza cases here in the U.S., it was more difficult to make the argument that the current paradigm using antibodies, did not in fact make sense (prior exposures meant that an unknown number of people would have a quicker antibody response time). But now, with COVID-19, and with the general population having absolutely no immunity to COVID-19, and yet with over 95% of patients recovering within 10-14 days, well before a substantial antibody response, it becomes glaringly and painfully obvious that the current paradigm is severely flawed. If in fact, the body isn't fighting off the virus as described by the current paradigm (with antibodies as the central player), but the body is still able to fight off the virus handedly in over 95% of cases, why wouldn't that very successful body mechanism to rid the virus be used in any further encounter with the virus? We are all amazed at antibodies and how well they work and protect us from illness. Yet, here is this most striking fact that antibodies aren't very involved in recovery for over 95% of COVID-19 patients. And still, do we really need to invoke antibodies as being the central player in a second exposure to COVID-19? If a patient only had mild symptoms with COVID-19 and recovered well (clearly without antibodies involved), can't that same person if re-infected with the identical same strain of COVID-19 also recover well the second time, even without antibodies? If we are all so impressed with antibodies and how amazing they are, shouldn't we be more curious what system the body is using to actually recover from COVID-19 and shouldn't we be even more amazed at that system? Because clearly that system is working for over 95 percent of COVID-19 patients and even more remarkable, that system requires no prior exposure to COVID-19. I would argue that the incredibly specific nature of antibodies pooled the wool over our collective eyes in regards to how we recover from respiratory viral illnesses.

The current COVID-19 pandemic gave us an incredible research study to show us the irrelevance of virus specific antibodies. Approximately 20 million people in the U.S. tested positive for COVID-19 in 2020. Within about a week, about 90% of these infected patients improved or were well on their way to recovery. Antibodies take at least 14 days to mount a significant response. The conclusion is difficult to challenge, that the majority of 20 million people eradicated the virus without significant virus specific antibodies present (the antibodies show up too late). In the past, with the flu, an argument could always be made that patients had some immunity to the current flu because there can be some cross-reactivity with antibodies from previous years. However, with this current COVID-19 pandemic, scientists and researchers are fairly certain that prior to infection, no one in the U.S. had a COVID-19 antibody and yet the majority of 20 million people who were exposed to COVID-19 were able to clear the infection well before COVID-19 antibodies formed in their bodies. How much more convincing evidence is required to show that COVID-19 specific IgG antibodies have at most a very minor role in the eradication of this respiratory viral infection?

Without being bound to any particular mechanism, the most likely mechanism of viral particle spread in the lower respiratory tract is as follows; an inoculating dose of virus reaches the lower pulmonary epithelial cells. The viral load bypasses or penetrates through the muco-ciliary defense overlying the epithelial cells. The virus particle attaches to the epithelial cell. Viral RNA is incorporated into the cytoplasm of the epithelial cell. Many viral particles are formed within the epithelial host cell. As the human cell lyses and breaks apart, many viral particles are released into the pulmonary tract airways and viral particles are moved to different areas of the lung via the muco-ciliary sweeping action and by macro actions such as coughing and sneezing. Other epithelial cells are then infected in a similar fashion. Repeated cycles of virus amplification and spread throughout the airways occurs via this mechanism. Tremendous build-up of virus specific antibody in the blood is unlikely to prevent any aspect of this mode of spread throughout the lung.

If we examine the virus-antibody fight purely from a mathematical perspective, it will be more discernible why the deciding battle between us and the virus has to be mostly within the cell. As an example, there are 1 million virus particles in the lung (for the sake of argument), and antibodies specific to the virus have been generated. The author does not believe these antibodies can neutralize even 10 percent of these virus particles, but to give the current paradigm the benefit of the doubt, we will assume the antibody can neutralize 95% of these virus particles. Now, 50,000 virus particles remain and infect other cells and each cell produces at least 100-1000 virus particles. We are now at 5 million to 50 million virus particles (from 1 million). That is with assuming antibodies have a 95% success rate at neutralizing the virus particle. We can make this argument even more convincing. At 99% efficiency of neutralizing 1 million virus particles, 10,000 virus particles remain from the starting hypothetical 1 million virus particles, and these 10,000 virus particles then infect cells and each cell produces 1000 virus particles. That leaves 10 million virus particles starting from a hypothetical 1 million virus particles, even with an antibody kill rate of 99%. Clearly, in this conflict between humans and respiratory viruses, the battle has to be within the cell. Preventing the virus particles from invading cells is impossibly difficult. That is not where the battle is. The battle is inside the cells. One-celled life forms have been fighting viruses for billions of years. Cells are efficient at clearing viruses.

The current paradigm of viral respiratory illness and the subsequent recovery revolves around antibodies that inactivate the virus. The inventor does not believe IgG antibodies are principally involved in the healing and subsequent eradication of respiratory virus infections. For example, in a hypothetical situation, a left upper lobe epithelial cell is infected by COVID-19. The virus replicates within the cell and over time hundreds to many thousands or more virus particles are released from the infected epithelial cell into the lumen of the airway (the epithelial cells line the inside diameter of the airway tube). If the current paradigm (antibodies are a principal player in the defense against respiratory viral infections) is correct, then since this patient has never been infected with COVID-19, first the virus has to replicate sufficiently within the lung cells and then some virus particles have to make it through the epithelial basal lamina layer, reach the blood vessel wall, breach the blood vessel wall, finally arriving into the blood within the lumen of the blood vessel. Then, lymphocytes began creating IgG antibodies specific to COVID-19. This isn't the end of the story. Now, these IgG antibodies that are in the blood stream have to make the exact same journey the virus particle made from the epithelial cell into the blood stream but in reverse AND a little further. To be precise, the antibody has to go through the blood vessel wall, reach the basal lamina layer, completely penetrate that layer, then the antibody has to either go THROUGH the epithelial cell (difficult to impossible) or BETWEEN the epithelial cells and finally reach the lumen of the airway which is where the thousands of virus particles have been recently released from the one infected cell. Tight junctions between epithelial cells may be looser during infection, but it is still quite a journey for these antibodies. Even if we assume the body ramped up its production of COVID-19 specific antibodies and there are now a billion antibodies in the blood, will enough of these antibodies penetrate the blood lung barrier and reach the airway lumen before these thousand recently released virus particles infect neighboring epithelial cells?

The blood gas barrier is an exceptional structure because of its extreme thinness and its extreme strength. The human pulmonary blood-gas barrier covers an enormous area. The tissue barrier separating air and blood consists of two cell layers: an epithelial layer facing the alveolar lumen and an endothelial cell layer facing the capillary lumen. The basement membranes of these cell layers are between these two cell layers. The basement membrane is comprised of type IV collagen and is extremely strong. The type IV collagen of the combined basement membranes provides the required tensile strength of the blood-bas barrier (BGB). It is well known that collagens are some of the strongest tissues in the human body.

In basic terms, the lung is like an air-filled balloon. Red blood cells and antibodies exist on the outside of this balloon membrane and lung epithelial cells are on the inside of this membrane. The lungs cannot work effectively if filled with fluid so obviously this “balloon” lung membrane is fairly watertight. How large is a water molecule versus an antibody molecule? For perspective, an antibody molecule size is 150,000 Daltons and a water molecule size is 18 Daltons. If the lung “balloon” membrane barrier can effectively prevent water molecules from passing through, why would that strong, tight barrier allow antibodies to easily pass through? If a water molecule cannot easily pass from the blood side of the lung to the epithelial cell side of the lung where the infections from the COVID-19 virus are taking place, how would an antibody molecule be able to traverse this “blood lung barrier”? This barrier concept is NOT new. Any upcoming COVID-19 vaccine assumes that a COVID-19 antibody formed in response to an intramuscular injection of a covid antigen results in a covid neutralizing antibody. For the antibody to neutralize a covid virus particle, the antibody must be at the site of the lung epithelial cell infection. HAS ANY RESEARCHER SHOWED HOW THE ANTIBODY PASSES THROUGH THE BLOOD LUNG BARRIER? I have not found a single published schematic showing the path of the antibody from the capillary to the alveolar epithelial cell surface. An antibody molecule is only effective if it can reach the surface region of the epithelial cells that are at risk for infection, which is on the OTHER side of the “blood lung barrier”. The blood brain barrier typically allows molecules less than 500 Daltons to pass through. Why would the blood lung barrier allow IgG antibodies which are 150,000 Daltons, to pass through easily?

There is a war raging inside a patient that is infected with a respiratory virus. The outcome of course is determined by many factors. Whether a patient recovers and overcomes the virus or not can be described in general terms numerically as follows; the rate of viral replication and spread versus the relative rate of uninfected neighboring cells being warned and creating a less hospitable environment for virus propagation by overall decreasing RNA translation into protein. The warning of neighboring uninfected cells is accomplished by spread of this factor. The therapeutic formulation of this invention is preparing the factor or combination of factors in various ratios, providing to a patient with a respiratory viral illness this formulation, to be inhaled as a mist and resulting in subsequent spread of the factor(s) throughout the lung. This inhaled formulation of factor(s) now signals uninfected lung cells at a much faster rate than occurs normally and hopefully faster than the rate of viral propagation and spread. Evolution evolved this simple and elegant method of signaling neighboring uninfected cells that a viral infection is underway and potentially spreading. If these factors are normally only present intracellularly, but when the cell dies, the intracellular contents (factors) are now released and present extracellularly, and the factors are able to alter surface receptors of neighboring cells resulting in decreased overall protein production in the signaled cells, use of the intracellular contents (factor) as a method of signaling surrounding uninfected cells that a nearby cell has been infected and died is a very efficient system. Lung epithelial cells throughout evolution were most likely were injured by infections. Throughout human history, lung epithelial cells were not substantially injured by falling, trauma, accidents, or physical blows. Lung epithelial cells throughout evolution most likely had to fight off viral and bacterial infections. It is impossible to imagine a more elegant or simple signaling system that informs neighboring uninfected cells that a neighboring cell has died (and predominantly of infection) wherein the signaling results in appropriate action by the cell to make virus propagation less likely (overall decrease in cell protein production).

Eukaryotic cells use DNA to code their genetic material. RNA is copied from the DNA. Proteins are made from the RNA. What is very notable in eukaryotic cells is the efficiency with which RNA within a cell is destroyed. No system is in place to destroy DNA. But RNA is degraded efficiently by many different enzymes. Imagine a cell that is infected by an RNA virus. The host cell uses the viral RNA and makes all the protein parts for the virus using this viral RNA. The rate of virus protein parts being produced may outpace the destruction of this viral RNA. Then the cell loses the battle. If protein production in a cell is significantly reduced (e.g. phosphorylation of eIF2α, which results in less translation of RNA into protein), then the outcome for the cell is determined by how quickly viral RNA is degraded by the many enzymes versus how quickly the viral protein parts are made using the available viral RNA. Now, we can shift this outcome in the cells favor by reducing overall protein production in the cell.

The invention of this disclosure promotes and helps the body use the strategies already in place to fight respiratory infections from RNA viruses. Humans have evolved from early life forms over possibly millions of years. Viruses have been on earth and all organisms that exist today, exist because they have co evolved mechanisms that can handle viruses.

The current invention does not require antibody production as a significant aspect of how the body overcomes a respiratory virus illness. Decreasing protein production within cells and signaling cells to decrease protein production is an effective means of overcoming viral illness. When there is overall relatively decreased protein production within a cell, but the rate of RNA degradation is substantially unchanged, the cell provides a harsher environment for viral replication. The outcome of a respiratory viral illness on an organ level (whether a patient recovers or becomes more sick) is determined by the relative rate of viral propagation in the lung versus the rate of signaling of healthy cells via a factor derived from damaged cells. The outcome of a viral infection on a cellular level is determined by the relative rate of protein production versus the rate of RNA destruction.

The most important aspects of the innate immune system in the defense against respiratory viral illness includes decreasing protein production, increasing ribonuclease activity, and signaling via cytokines and other damp/pamp molecules. Neutrophil recruitment and release of proteases from neutrophils is not beneficial. Activating the innate immune system while also diminishing white blood cell movement and white blood cell release of proteolytic enzymes is the goal. Activation of the innate immune system is effective against respiratory viruses principally because of the global decrease in protein production effected by the phosphorylation of eIF2α.

Disclosed is a method for treating a respiratory viral infection by enhancing the immune response. The purpose of the enhancement is to ultimately decrease global protein production within both infected and uninfected cells. More specifically, the method described activates the final common pathway of phosphorylating eIF2α, which leads to global suppression of protein production. It is also important to decrease or limit white blood cell movement, migration, and protease release at or around the time of the immune response enhancement. The method comprises administering the factor or combination of factors (COF) to enhance the immune response which leads to phosphorylation of eIF2α to a subject after the subject has been exposed to a respiratory virus or after the subject has been infected with the respiratory virus. The factor or combination of factors is preferably administered at the time of exposure or at the time the subject has been infected or within 3 days of exposure or infection with the respiratory virus. The factor or combination of factors (COFs) are discussed in detail in other parts of this disclosure. The COFs are preferably administered at the time of exposure or at the time of viral illness to about 4 days after onset of viral illness. Most preferably, the COFs are administered between the time of onset of the viral illness to 2 days after onset of viral illness. In a preferred embodiment of this method, the COFs is administered via an aerosolized route or via intranasal, dermal, intramuscular, or intravenous route. This method activates the innate immune system and can be used to shorten the duration of the respiratory viral illness and decrease the severity of the respiratory viral illness.

The invention provides an unconventional approach to activating the innate immune system and the integrated stress response using a factor or factors (these factors are described throughout this disclosure). The main goal of activation of both the innate immune system and the integrated stress response is to induce decreased protein production within cells and to increase the relative rate of ribonucleases within cells. Achievement of this main goal in both infected and uninfected cells produces the “anti-viral” state and limits the severity and duration of the subjects' viral illness. Activating the innate immune system also mobilizes white blood cells including macrophages, neutrophils, and lymphocytes. Often, in severe viral respiratory illnesses, the subjects' white blood cell migration into the target tissue and subsequent activation causes more damage through the release of proteolytic enzymes. The goal in respiratory viral illnesses is to activate the integrated stress response primarily to increase phosphorylation of eIF2α which reduces protein production within cells and inhibits viral mRNA translation into protein. Since activating the innate immune system may also mobilize white blood cells, providing a medication or therapeutic that decreases white blood cell movement and decreases white blood cell release of proteolytic enzymes is useful either slightly prior to, during, or slightly after activating the innate immune system. Medications or therapeutics to limit white blood cell activity are described elsewhere in this disclosure.

The insight of this disclosure is the discovery that the most important mechanism to overcome most viral illness is to 1) relatively increase the activity of ribonucleases, 2) decrease protein production within cells and 3) signal other cells and warn of a virus infection. Other parts of the immune system may be more damage inducing than beneficial. For example, neutrophils and release of their proteolytic enzymes are what probably effect lung damage and cause “white out” on chest x-ray. Activating the innate immune system initiates various different pathways that are helpful against pathogens. However, activating white blood cell migration and white blood cell release of proteolytic enzymes is not beneficial against viruses. The human body has amazing defenses against viruses. But without a clear understanding of the main mechanisms the body uses against viruses, facilitating the body in its fight against viruses is difficult.

### Introduction to Four Strategies

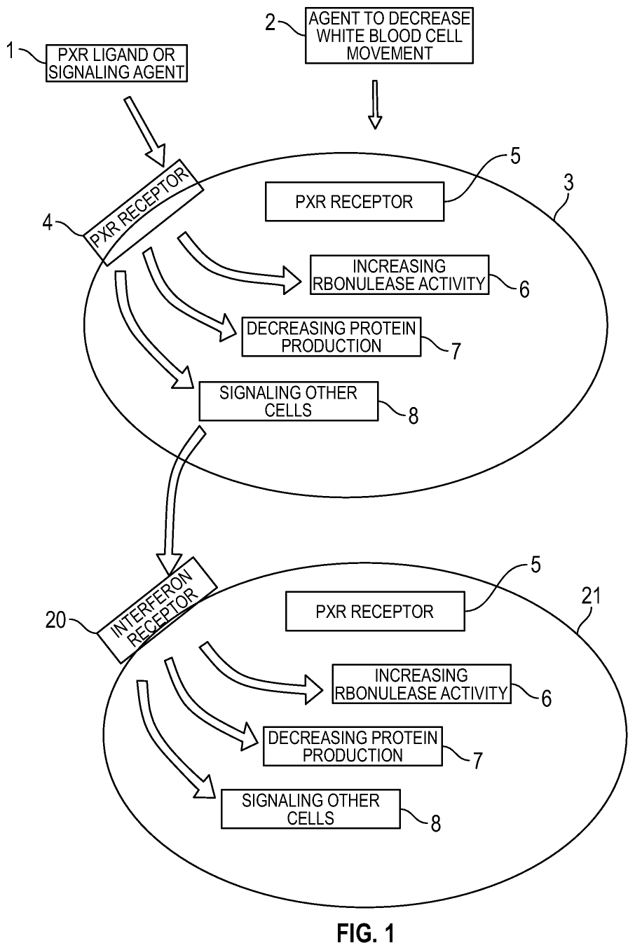

Referring to FIG. 1, a PRR ligand or signaling agent 1 binds to a membrane PRR receptor 4 and well-known pathways result in increasing ribonuclease activity 6, decreasing protein production 7, and production of signaling cytokines 8. The signaling cytokines 8 from the cell 3 interacts with receptors 20 on a second cell 21 and that binding results in increasing ribonuclease 6, decreasing protein production 7, and production of signaling cytokines 8 in the second cell 21. Still referring to FIG. 1, Block 1, a signaling agent, refers to factors or agents discussed under the third fundamental strategy. Block 2, an agent to decrease white blood cell movement, refers to factors or agents discussed under the fourth fundamental strategy. Block 6, increasing ribonuclease activity, refers to factors or agents discussed under the first fundamental strategy. Block 7, decreasing protein production, refers to factors or agents discussed under the second fundamental strategy.

The strategy the host uses, whether mammal or plant or human, to detect the presence of a pathogen depends on the type of pathogen. Determining “host” molecules versus “non-host” molecules is critical in the ongoing battle against pathogens. For example, virus detection by the body is typically more difficult than bacteria detection because viruses must enter the host's cells in order to replicate before leaving the cell and infecting other host cells and so viruses are present intracellularly for a much greater percentage of the time, relative to bacteria. Viruses generally do not have protein making machinery and rely upon the host's protein making machinery to translate its RNA into virus proteins. It makes logical sense then, that the host's virus detection systems and mechanisms will also be much more likely to be intracellular than extracellular. A human host can be exposed to a virus and within minutes to hours, the virus can have entered the host cells. Although evolution is not perfect, it generally finds the most efficient solution. Expending resources to scan for viruses extracellularly would be highly inefficient, given the limited amount of time viruses spend extracellularly once a host is inoculated to the time the virus is able to penetrate the host's cells. As expected, the innate immune system's methods of virus detection include “sensing” the presence of double-stranded (ds) RNA which is not part of the host's cellular mechanism or processes. The cell's detection system for “sensing” ds-RNA is generally found within cells, since that is the location of viral propagation and ds-RNA is usually found intracellularly for that reason.

The host defense against viruses is akin to warfare. The applicants' approach to defeating the virus is similar. 1) destroy the enemy (ribonucleases), 2) know what the enemies' goals are (viral RNA wants to become protein) and thwart the enemy, 3) signal allies that an invasion is underway (interferon), and 4) use military tactics/weapons that hurt the enemy while minimizing harm/damage to allies (decrease white blood cell release of damaging agents or materials or substances, since white blood cells are not critical to the fight against viruses).

Following detection of a virus pathogen, the basic defense strategy against viruses is already in place. Ribonucleases are enzymes that are very efficient at degrading RNA, all RNA including virus RNA and human RNA. Knowing that the virus RNA is essentially completely dependent on host protein-making machinery to initially translate the virus RNA into protein, the second integral strategy to fight a viral invasion is to globally decrease protein production within cells. This is also a very basic strategy, as viruses initially have no protein making machinery and need their DNA or RNA converted into virus proteins which help replicate the virus, helps defend against the host, and ultimately aids in migration out of the cell so the virus can infect other cells. As described above, two of the host's fundamental strategies against viral attacks are both intracellular.

The host's third fundamental strategy against viruses is to communicate or warn or signal other host cells that there is a virus infection underway. Once the other cells (healthy or infected) are warned that there is a virus infection in the host, the warned cell can increase relative ribonuclease activity and/or within the cell globally decrease protein production. The warned or signaled cell can also produce cytokines that warn other cells. The signaling from the host infected cell to the other cells can be accomplished with cytokines and/or interferon molecules. Pattern Recognition Receptor (PRR) agonists initiate the signaling by binding to PRRs on a cell and this binding of the PRR agonist to the PRR initiates pathways that include cytokine and interferon production. There is also overlap with other DAMP and PAMP molecules. Often, because the beginnings of virus infections in the host are often subtle (not within the cell, but outside the cells that are infected), detection of virus infections is more difficult than the detection of viral infections. For example, once a cell is signaled by binding of interferon to the cell's interferon receptors, it induces release of intracellular enzymes such as 2′-5′-oligoadenylate synthetase. This 2-5A synthetase uses ATP to synthesize 2′-5′-oligoadenylates, which activate latent ribonuclease (RNASEL), resulting in a less favorable environment for virus replication. Interferon stimulates many genes (ISG) including PKR (protein kinase R). Activated PKR results in the phosphorylation of eukaryotic initiation factor 2 on its a subunit (eIF2α), a major regulator of the initiation phase of mRNA translation, the rate limiting step of protein synthesis.

Because a virus infection can be more difficult to detect, once the host detects a virus infection, the viral infection can already be progressing and so quickly warning other cells of a potential infection and rapidly amplifying the warning signal can be critical to prevent morbidity. Due to the subtle nature of virus infections, and the serious consequences for a late host response, once a virus infection is detected, the host warning systems are often easily triggered and rapid even to the slightest stimuli, and so there can be host responses to “false” warnings. Given that a too slow and/or too late host warning system (for a viral infection) can result in host death whereas the consequences for an early unnecessary “false” warning (in the situation where there was no virus infection), are only a slight set-back for the host, it follows that the average host will probably have evolved a warning system that is set to overcall a virus infection (false positive). In evolution, the costs of “false negatives” are much higher than “false positives” on survival. During human evolution, hearing the grass rustling in the savanna even if was due to the wind and concluding that is was a tiger (false positive) cost much less than concluding that the rustling sound was the wind, if it was due to a tiger (false negative); false negative conclusions can be fatal. False positive conclusions are much less costly. We can conclude that virus detection is much more difficult for the host than bacteria detection. The human body will generate more “false positives” in detecting viruses and defending against viruses than how the host deals with bacteria. Because of this sensitivity in the human body's general reaction against viruses, a variety of stresses result in cellular activation of PKR (protein kinase R) and the ensuing global decrease in protein production within the cell (one of the fundamental hosts' strategies against all viral infection).

Viruses relative to bacteria spend much more time within the host cell. Bacteria, even after invading a host, rarely enter the cells of the host. The molecular patterns unique to bacteria are more easily detected by the host compared to viruses which spend a greater degree of time within host cells and bacteria detection mechanisms are more likely to be present on cell surfaces. Virus detection mechanisms by the host are more likely to be present intracellularly.

Each fundamental strategy the body uses for viral infections can be facilitated. These are the three fundamental strategies, 1) Increasing the relative amount and or activity of ribonucleases, 2) decreasing protein production within cells and 3) signaling cells that there is a viral infection so that those signaled cells can increase the relative activity of ribonucleases and/or decrease the rate of protein production, which results in a less hospitable environment for viruses within the cell. The invention of the current disclosure is the facilitation of each fundamental strategy, in conjunction with one and/or two of the other fundamental strategies, or in various combinations of facilitation.

The strategy of signaling (the third fundamental strategy) will be discussed at length. In warfare, an early warning system to notify the leadership of an enemies' presence drastically increases the chances of success for almost any response to the enemies' presence. When a virus infects a host, it is similar to a war. Each infected cell and potentially infected cell is a separate battleground. Early signaling of uninfected cells prior to invasion by a virus increases the odds that the cell will win the battle. The two fundamental strategies against viruses (relatively increasing ribonuclease activity and relatively decreasing protein production) can be implemented prior to the virus injecting viral DNA or RNA into the cell when signaled in advance.

Interestingly enough, the activation of TLR-4 (toll receptor 4) via LPS or similar ligands also increases the production of interferons. This shows that although host defense strategies against bacteria and viruses are quite different, there is overlap in the response against pathogens. It is easier to facilitate the human body in its recovery against a viral illness once the true nature of the mechanisms behind the healing are known. The massive attempt by the U.S. government and private companies to create a COVID-19 vaccine shows the inefficiency and wastefulness of both time and resources when proceeding under an untested or false assumption. Again, 20 million Americans tested positive for COVID-19 in 2020 and well over 90% had recovered or were well on their way to recovery by 7-10 days. IgG antibodies require at least 2 weeks from good exposure to generate “therapeutic” levels in the blood. Since it is highly unlikely that IgG antibodies were significantly involved in the human bodies defense against COVID-19, the three outlined fundamental strategies are the definitive mechanisms involved in the “healing” from COVID-19. Once the true mechanisms the body uses in recovering from COVID-19 are understood, it is infinitely easier to facilitate the body as it “heals” from COVID-19. Although the disclosure mostly targets the treatment of respiratory viral infections in humans, the combined strategies will be beneficial in treating many viral illnesses that affect mammals.

In the invention of the current disclosure, the mechanism the host cells use to combat viruses are facilitated. Many of the mechanisms cells use to detect virus pathogens are often intracellular. If we are to facilitate the host cells in their fight against viruses, delivering a method to activate the host cells detection system for viruses will be useful. However, since many of the classic host cells methods to detect viruses are intracellular, delivery of a therapeutic to activate intracellular host virus detection mechanisms is more complex. Delivering a therapeutic to the surface of cells is much easier. Activation of the PRRs on cell surfaces is easier and the resulting signaling cascade generally overlaps with the bodies mechanisms in combatting viruses. As an example, LPS (a bacterial cell wall component) binds to TLR4 and ultimately the host cell produces interferons also, which are released and signal other cells that a virus infection is underway. The TLR4 receptor is located within the host cell membrane and LPS present extracellularly can still bind to TLR4 and induce host cell production of interferon. But, other cytokines are also produced by the host cell including cytokines that mobilize white blood cells and activate white blood cells. Combining an agonist for PRRs with a therapeutic to decrease white blood cell migration and/or activation is important in limiting the activation of the parts of the immune system more effective against non-viral pathogens. This is further discussed under the “Fourth fundamental strategy” in this disclosure.

Most current medical treatments for respiratory illnesses are directed at treating the virus replication itself directly. But, understanding how the virus replicates and then spreads to other cells, preparing other neighboring cells is important. Humans try to create medicines to fight the virus. It should be remembered that eukaryotic cells have been fighting these viruses for millions of years. Currently drug development strategies for RNA respiratory viruses focus on vaccines (methods to increase virus specific antibody production) or drugs that target specific proteins within the viral genome. Drugs that target specific viral proteins will have to undergo lengthy toxicology assays and human studies will have to be followed for side effects for a lengthy period. The human body currently has many mechanisms to fight virus replication and these mechanisms have evolved over hundreds of thousands of years and some of the more basic mechanisms may have been present for millions of years, with the beginning of eukaryotic one celled life forms. The strategy of the invention of this disclosure is to facilitate the already present strategies the cells have incorporated. This general reduction of protein synthesis within the cell can drastically reduce the ability of the virus to multiply and propagate within the “warned” cell. This method is not nearly as specific against individual respiratory viral infections. Lack of specificity in targeting a particular viral infection in no way implies less efficacy against that particular virus.

### First Fundamental Strategy

Ribonucleases (RNases) are a very ancient and important class of enzymes. RNases belong to a very important family of proteins that have been extremely well studied. They were once believed to have only the primary function of catalyzing nonspecific RNA turnover and degradation but now are recognized to have a broad spectrum of activities including antiviral, immunomodulatory, and angiogenic roles.

Within the signaled cell, the greater battle looms and the outcome for each cell is determined by the rate of RNA degradation versus the rate of protein formation. Simply put, once a viral RNA enters a cell, if RNA degradation occurs at a normal rate, but protein production is relatively reduced, the viral RNA is degraded before substantial viral protein parts can be translated from its RNA and the cell overcomes the viral intrusion. Regarding RNA degradation, since cells transcribe more RNA than they accumulate, researchers discovered early on that active methods for RNA degradation exist. In all organisms tested from all kingdoms from the tree of life, the efficiency of RNA degradation within the cell is notable. The substantial similarities in the method of RNA degradation between the different life forms underline its importance. RNA degradation enzymes (ribonucleases) are present everywhere which is a vital clue to the importance of these enzymes. The author's hypothesis that the evolution of enzymes that degrade RNA in DNA based cell structures occurred because of the existence of RNA viruses (the need to destroy foreign RNA) allowed the increased complexity of almost all feedback loops involving proteins. In essence, it was the RNA virus that created the condition that forced the original DNA cells to create an RNA destroying enzyme. The ribonuclease was the result of that interaction and the ribonuclease is now a master “off” switch for all enzymes and proteins that derive from RNA. It was the original and only defense against RNA viruses for those primordial cells that used DNA to encode information. Since the origin of DNA based life, why would the fundamental method of combating RNA viruses NOT still be used now?

The most likely cause of death of a lung cell in a healthy individual is infection. If signaling to the surrounding cells result in less RNA translation and less protein production compared to the amount of protein production in a healthy state, then the cell can reduce viral propagation and this produces an “anti-viral” state within the cell. If the signaling results in surrounding healthy cells producing less protein, then the neighboring cells have a huge advantage over the first infected cell that hasn't lowered its protein production as quickly. Evolution is extremely efficient. Imagine early in mammalian evolution. Respiratory viral illnesses kill large numbers of people efficiently. The survivors happen to have cell receptors to intracellular factors and the cell receptors to these intracellular factors that are now floating around extracellularly, cell receptor activation results in decreased protein production via currently well-known pathways. This would be an extreme advantage for survival. Viruses have been with us for millions of years, probably since the beginning of biological life. Again, the outcome of viral respiratory illness for an organism is determined by the relative rate of viral propagation versus the rate of infected/injured cells warning neighboring cells and those healthy neighboring cells preparing themselves for viral invasion by reducing overall protein production within the warned cell. The outcome of respiratory viral illness is determined by how quickly cells are signaled/warned and prepped prior to viral invasion. Furthermore, on a cellular level, the outcome is determined by the relative rate of RNA degradation (via ribonucleases) versus rate of protein production. The larger this ratio, the less protein produced and less chance of viral propagation.

RNase L is an interferon-induced nuclease that, upon activation, destroys any RNA within a cell. Ribonuclease Inhibitor (RI) is a 50-kDa cytosolic protein that binds to ribonucleases with femtomolar affinity and renders ribonucleases inactive. The binding of RI and its target ribonucleases are among the tightest known in biology. Oxidation of RI is one mechanism by which the activity of RI is regulated in the cell. The ability of ribonuclease to manifest its catalytic activity in the cytosol is related to the concentration of RI in the cytosol. RI is only able to inhibit the function of ribonucleases when the RI is in a reduced state. Oxidative stress within the cell also favors oxidation of RI and oxidized RI renders it much less effective in inhibiting ribonuclease activity. Upon oxidation, RI undergoes rapid unfolding and inactivation, subsequently releasing bound ribonuclease. With many viral infections, there is often a stimulation of the oxidative stress response and an increase in ROS (reactive oxygen species). Oxidation of the reduced RI prevents the RI from binding to the ribonuclease and the now free ribonuclease is able to destroy RNA, including viral RNA.

Reactive oxygen species (ROS) production within the cell is induced upon nutrient deprivation (glucose and/or amino acid deprivation). The increase in oxidative stress within the cell increases the relative rate of ribonuclease inhibitor oxidation which frees up ribonucleases to degrade cellular RNA. Elimination or drastic reduction of calorie intake during the initial phases of a viral infection is helpful in overcoming the virus infection since pathways that increase ROS production within cells is very well established and a relative increase in ROS within cells increases the oxidation of RI which then releases ribonucleases which are then more available to degrade RNA including virus RNA.

It is likely to be found true later that decreasing caloric intake during the initial phases of a COVID-19 infection (first few days) is much more likely to be much more beneficial to survival than either hydroxychloroquine use OR COVID-19 vaccine implementation and yet no health authority to date worldwide has mentioned this (that the inventor is aware of).

Decreasing caloric intake drastically will notify the body that this is NOT the time to build new proteins (of course exceptions will occur). Nutritional deprivation is well known to induce a relative increase in cellular ROS. There are many well-known pathways to decreasing protein production within cells when cells are even slightly nutritionally deprived and those pathways also will contribute to reduction of virus RNA translation. IV fluids given to virus infected patients should avoid glucose to maximize the chances of patient recovery. This very important behavioral adjustment (eating less) in the recovery of respiratory virus illness is well conserved. Of 7 billion people on earth, it can be easily argued that over 99% of us, as infants who were infected with a virus, became fussy and ate much less. Any behavioral trait that is that well conserved among humans is there for a very good reason.

### Second Fundamental Strategy

Decreasing RNA translation. Once a patient develops a respiratory viral infection (or any viral infection), a very important factor to improve the survival of the host is to decrease or eliminate protein from the diet for days to a week or two. Eliminating protein from the diet for that short term period will cause minimal harm to the host but will drastically impact the ability of the virus to replicate. Decreasing total caloric intake is also beneficial; nutritional stress activates Protein Kinase R that causes decreased global protein production within cells. Decreasing total caloric intake by 50% to 100% for a few days to a week will increase the probability of overcoming a viral infection significantly.

Going back 4 billion years when the first primordial DNA cell existed, even those first primordial DNA cells most likely had to battle RNA viruses. A most efficient strategy was to decrease protein production. That strategy is still implemented today by all cells and by most organisms. That strategy is even implemented by cells for different stresses. Whatever the form of the stress, most cells quickly stop making proteins when under pressure.

In the invention of this disclosure, it has been repeatedly emphasized that a one component in the cells defenses against the RNA virus includes reducing overall protein production. The molecular mechanism achieving reduction of protein production in the cell is summarized here. Because of its direct contact with the outside world, the lung is continuously challenged by inhaled insults (e.g. chemical, smoke, infectious agents), all of which trigger various cellular stress pathways. Phosphorylating the a subunit of eukaryotic translation initiation factor 2 (eIF2α) plays a critical role in these stress pathways. This enzyme, eIF2α, is essential for almost all translation of RNA into protein within the cell and when eIF2α is phosphorylated, it is inactive, which results in less protein production within the cell. This single reaction is the common pathway to integrate signaling from multiple surface and cytoplasmic stress sensors and so has been named the “integrated stress response” (ISR).

In response to various stresses, mammalian cells initiate a common pathway, termed the integrated stress response (ISR), to restore homeostasis within the cell. The single most important aspect of this pathway is a decrease in cellular protein synthesis and the induction of several genes including the transcription factor AFT4. Decrease in cellular protein synthesis is accomplished by phosphorylation of eukaryotic translation initiation factor 2 alpha (eIF2α). The final common pathway for all stress stimuli that activate ISR is phosphorylation of the alpha subunit of eukaryotic translation initiation factor 2 (eIF2α) on serine 51. There are at least 4 activated kinases in response to stress that converge on phosphorylation of eIFα. PERK (PKR-like ER kinase), PKR (Double-stranded RNA-dependent protein kinase), HRI (Herne-regulated eIF2α kinase), and GCN2 (General control non-depressible protein 2) kinases are all activated by stress within a cell. Cellular decreased protein synthesis is accomplished by phosphorylation of the a subunit of eIF2 on serine 51, which converts eIF2 from a substrate into an inhibitor of eIF2B. Phosphorylated eIF2α binds more tightly to eIF2B and blocks its GEF activity and thus reduces general translation. eIF2α is a major regulator of initiation of mRNA translation, which is the rate limiting step of protein synthesis.

The eukaryotic PKR is generally activated by double-stranded RNA. Protein kinase R (PKR) is a serine-threonine kinase and plays a major role in cellular processes such as mRNA translation. Viral replication within mammalian cells require the production of double-stranded RNA and since double-stranded RNA is not normally present in eukaryotic cells, the presence of ds-RNA is a clear signal of an intracellular RNA virus presence and a very effective antiviral mechanism is to decrease protein production, as viruses are completely dependent on eukaryotic cells protein translation ability to propagate. A viral infection is detected by the presence of ds-RNA, the ds-RNA activates PKR, PKR phosphorylates eIF2α. The phosphorylation of eIF2α results in decreased protein production within the cell and allows more time for the ribonucleases within the cell to degrade the viral RNA. PKR can also be activated by other factors including heparin. Heparin, since it activates PKR, can be a very effective anti-viral medication.

The main effect of the ISR is on protein synthesis. The repression of cellular mRNA translation promotes cell survival during a viral infection since there is a reduction in viral mRNA translation. Amino acid depletion can also activate ISR by GCN2 and so this is another important mechanism that the individual can facilitate by consuming less to no protein during a viral infection. Eliminating protein consumption for a week will improve the body's ability to overcome a viral infection by providing less amino acids which results in less viral mRNA translation into viral proteins and also by activating GCN2 which results in phosphorylation of eIF2α and again less viral mRNA translation into proteins necessary for viral propagation within a cell. Reducing total caloric intake by 50% to 100% during the first few days of viral infection can also facilitate decreased viral propagation because the body will convert even available amino acids into fuel resulting in less amino acids available for mRNA translation into protein. This is a stark illustration demonstrating the utility of understanding the body's defense against a viral infection. Well before even language development and well before we understood the germ theory of disease, mammalian young responded to viral infections by consuming less food. Of 7 billion humans alive today, 99% of us, as infants, when infected with a virus had loss of appetite. The conservation of this pathway is strong evidence of its extreme utility in battling viral illness. Yet, there is not a single doctor that has stated the importance of this on a major news network in regards to COVID-19. A true understanding of the main mechanisms the body uses to overcome a respiratory viral illness leads to a correct way to facilitate the body in its efforts at recovery from the viral infection.

Phosphorylation of eIF2α can be stimulated by activators of eIF2α kinases such histidine, asparaginase, halofuginone, arginine deiminase, and many other known activators. This disclosure discusses many other factors that result in decreased protein production within a cell with most factors ultimately leading to decreased protein production via relative increase in phosphorylation of eIF2α. Salubrinal is another small molecule that maintains eIF2α highly phosphorylated, reducing protein synthesis. Guanabenz and Sephin 1 can also enhance the integrated stress response and limit protein synthesis and thereby prevent viral propagation.

A substance that stops or slows the growth or proliferation of cells by interfering with the processes that directly lead to the formation of new proteins (inhibitor of protein synthesis) is also a method of facilitating the hosts' defense against virus infection. Branched chain amino acids such as leucine, isoleucine, and valine are known to stimulate protein synthesis. Blocking those amino acids decrease protein synthesis also. Blocking of growth hormone can also reduce cellular protein synthesis which can help decrease virus RNA translation into protein.

PTEN, P53 can decrease mTORC activity which can decrease cellular protein synthesis. Many cancer medications suppress tumors by decreasing protein synthesis and these are also useful for a short term to decrease virus propagation. Several forms of cellular stress can trigger the repression of cellular protein synthesis.

Protein synthesis is controlled by the efficiency of the translational apparatus, which is determined by the factors that influence translation initiation (Kaufman, 1994). Initiation of translation involves consecutive recruitment of the small and large subunits of ribosomes to specific mRNAs, with the formation of an active ribosome at the initiation site. The predominant mechanism for control of protein synthesis appears to be reversible phosphorylation, under the control of selected protein kinases and protein phosphatases. The targets of covalent modification in this case are translational components, specifically the initiation and elongation factors (Hershey, 1991). The eukaryotic Initiation Factor 2 alpha (eIF-2α), which promotes the binding of initiator tRNA to the 40S ribosomal subunit, is an example of a factor that is regulated in this manner. Phosphorylation of eIF-2α is correlated with inhibition of protein synthesis in a range of eukaryotes (Rhoads, 1993)

Cells tailor protein synthesis so that proteins required for optimal conditions versus proteins that are necessary for stressful conditions are produced at the appropriate times. Genes that encode proteins involved in growth and proliferation use common codons. Genes that encode stress response proteins use rare codons. Cellular stress skews the tRNA pool towards those that recognize rare codons and increase the level of stress response proteins that are produced.

Decreasing food intake results in decreased mitogenic hormones and ultimately decreased cellular global protein synthesis. It is interesting to note that some over the counter “cough medicines” incorporate a zinc supplement. In fact, zinc is necessary for the adequate secretion of growth hormone from the pituitary and increased zinc facilitates the secretion of growth hormone which increases protein translation and is not helpful to inhibit virus growth. Insulin is also a growth-like hormone. Decreasing caloric intake decreases the level of insulin in the body.

Cells experience stress in many ways. Typically, no matter what the cause of the stress, cells quickly stop making proteins. Protein synthesis typically is controlled at the initiation of translation. But, in the protein synthesis phase of elongation, protein synthesis can also be halted. Chaperone molecules help partially formed peptides emerge from ribosomes. Under stress, less chaperones are typically present and if not present to assist nascent peptides emerge from the ribosome tunnel, less fully formed proteins are produced. Chaperone inhibitors can also decrease protein production including the production of virus proteins from virus RNA or DNA.

Within cells, the majority of intracellular proteins are degraded by the ubiquitin (Ub)-proteosome pathway (UPP). This pathway includes concerted actions of enzymes that link chains of the polypeptide co-factor, Ub, onto proteins to mark them for degradation. Following tagging, the 26S proteasome (which is a large protease complex) recognizes the ubiquitinated proteins. Relative increased activity of this UPP may be beneficial in cells that have been infected with a virus. TGF-β regulates many cellular process and is one of the most potent inhibitors of normal cell growth and also protein synthesis in cells.

RNAi (RNA interference) refers to a phenomenon in which small sections of RNA can prevent protein translation by binding to the messenger RNAs that code for those proteins. Increasing the RNAi for viral proteins can also decrease virus protein production. There are two types of RNAi, small interfering RNA (siRNA) and MicroRNA (miRNA). Both are sections of RNA of approximately 22 nucleotides in length.

Without being bound by any particular mechanism, the general principle is that increased global cellular protein translation is a pro-virus propagation environment and that a decreased global cellular protein translation environment is less favorable for virus propagation. Growth hormone, a protein hormone of about 190 amino acids has direct effects and indirect effects. The indirect effects of growth hormone are primarily mediated by a IGF-1 (an insulin-like growth factor), a hormone that is secreted from the liver and other tissues in response to growth hormone. There are nine essential amino acids that the human body does not synthesize, so they must come from our diet. It turns out that phosphorylation of eIF2α is a conserved response from yeast to mammals that inhibits overall translation initiation, and it is an output of anti-mitogenic signals.

An overall relative decrease in protein production is a very well-defined strategy for many reasons in the evolution of various life-forms. Any and every life form has these issues and good management of available resources provided a significant advantage compared to a life form that was not able to manage available resources well. For example, during early human evolution, individuals were often hungry, given limited food. When food was limited, limiting growth and protein production while reserving caloric intake for activity (moving) and thinking (the brain has a high caloric demand) was critical. An individual that did not manage limited food well but tried to grow when food was limited meant that the individual did not have sufficient calories for activities such as hunting and was much less likely to survive. Since a multi-organ individual has billions of cells that need to be informed of whether to “grow”, an anabolic state, or to reserve calories for activity, an efficient signaling system to inform individual cells how to allocate calories was critical. Hormones played a critical role and cortisol is a perfect example. Corticosteroids are known as “stress hormones”. It is incorrectly described. It should be more correctly described as the “awake hormone”. Being awake and moving and thinking was “stress” for evolving humans prior to even only the past few thousand years. While awake, most caloric available energy should be diverted to the systems necessary for muscle movement (activity) and brain power. That gives the individual the highest chance of surviving. Cortisol aptly spikes in the morning and levels drop at night. Cortisol signals cells to decrease protein production and increases gluconeogenesis, as one would expect for better allocation of limited resources. There are many cellular mechanisms in place to effect this transition from an anabolic to steady state to a catabolic state and back. Most carbon based life forms have mechanisms in place to adjust for “survival” versus “growth”. It turns out that one of the most fundamental and efficient ways to “fight” viruses is to be in a relatively less “anabolic” state. When a virus infection is underway, why would the human body NOT use the pathways in place that convert the cells into a less “anabolic” state? As can be seen, this paragraph would have been perfectly appropriate under the “Third fundamental strategy” which is signaling other cells that there is a virus infection underway since cortisol is an incredibly quick signaling molecule but this paragraph would also have been perfectly appropriate under the “Fourth fundamental strategy” which discusses corticosteroids. Evolution is incredibly efficient and although there is redundancy, it will be clear to the reader of this disclosure that there is tremendous overlap in signaling cells to go from an anabolic to catabolic state (or the reverse) during times of nutritional deprivation and the bodies signaling of cells during a virus infection. One of the mechanisms used to switch from growth to energy conservation and vice-versa is discussed in the paragraphs below.

ROS were discussed under the First fundamental strategy. However, many of these factors overlap and affect all the fundamental strategies described in this disclosure. The process of protein translation constitutes the last and final step, the assembly of polypeptides based on information encoded by mRNAs. Protein translation is tightly controlled in a cell and protein homeostasis (proteostasis) is extremely important in cellular homeostasis and cell growth. During evolution, evolved pathways are not necessarily duplicated when serving a similar function. For example, the body's response during periods of food deprivation is similar to how the body responds during a viral infection since decreased mRNA translation is an extremely efficient method to limit virus propagation. It is well known that food deprivation causes a relative increase in cellular ROS. Although ROS are potentially harmful to cells because of their ability to oxidize cell components, at low levels, this ability allows ROS molecules to play a critical role in a variety of signal transduction pathways. It is often stated that when ROS levels exceed the functional capacity of antioxidants, a condition called “oxidative stress” is created. “Naming” this condition is misleading because antioxidants include many functional proteins that have a changed function on their oxidation status, such as the ribonuclease inhibitor (RI). When certain groups on the RI are oxidized, the RI does not inhibit ribonuclease. It is misleading to believe that once the functional capacity of antioxidants is exceeded, “oxidative stress” is created. This statement would only lead one to ask when the capacity of antioxidants is exceeded, since almost any part of the cell can be oxidized. Also, prior to this artificial capacity of antioxidants that is exceeded, many antioxidants such as RI are oxidized and the functional state of the cell has been drastically altered. To believe that there is no functional change in the cell just because the “capacity of antioxidants” has not been exceeded is to not realize (as an example) that 20% of RI being oxidized is very different from 90% of RI in the cell being oxidized, although the functional status of the cell in both cases would be drastically different and although both cases fall under “not exceeding the functional capacity of antioxidants”. Tightly regulating growth versus maintenance for a cell was extremely important in survival since cell growth during nutritional deprivation was much more likely to result in inappropriate allocation of valuable resources and could lead to cell death. It so happens that one of the best strategies in overcoming a virus infection is to decrease mRNA translation into protein and proteostasis is extremely well regulated since growth and survival depend on this ability to manage protein production appropriately. Nutritional deprivation (fasting) uses the bodies well regulated method of managing protein production to create an anti-virus environment. ROS are relatively increased during periods of nutritional deprivation and ROS targets many of the stages of protein translation, ultimately relatively decreasing protein production. There are many mechanisms whereby nutritional deprivation causes increased ROS and ultimately global repression of protein translation. Endoplasmic reticulum (ER) stressors such as ROS leads to ER stress, PERK/Ire1 activation, and eIF2α phosphorylation, which prevents initiation of translation. ROS induced suppression of protein synthesis is accomplished via a myriad of pathways and mechanisms and is extremely useful in the intracellular defense against viruses. ROS can also activate NF-κB signaling and downstream inflammatory gene transcription.

### Factor Discussion and Third Strategy, Signalling

The subject invention of this disclosure concerns a method of inhibiting a viral infection within a patient by decreasing the level of protein translation within a cell via a factor (or factors) interacting with cell surface receptors. As described herein, the most important characteristic of factors is the ability to substantially induce the cell stress response by interacting with cell surface receptors and ultimately inducing phosphorylation of a subunit of eukaryotic translation initiation factor 2 (eIF2α) so that protein synthesis within the cell is relatively diminished. In one aspect, the subject invention concerns a method of treating or ameliorating a virus infection within a patient by triggering surface receptors with factors that results in phosphorylation of the initiation factor and concomitant less protein production within the cell. Preferably, the methods of the present invention do not involve administration of factors that have been optimized for entry into the cytoplasm of the cell. The methods and compositions of the present invention are directed to surface receptor triggering by a factor or combination of factors that leads downstream to increased activity of protein kinase R and phosphorylation of initiation factor. The factors have the common essential characteristic/function of interacting with pattern recognition receptors followed by phosphorylation downstream of the a subunit of eukaryotic translation initiation factor 2 (eIF2α) so that protein synthesis within the cell is relatively diminished. The characteristics of these factors are further discussed at length because different categories of molecules accomplish the same essential function of interacting with surface cell receptors leading to relatively diminished protein production. The word “factor” is mentioned many times in this disclosure and refers to a molecule that binds to a type of pattern recognition receptor and binding of that molecule to the pattern recognition receptor leads to decreased protein synthesis within the cell.

In one embodiment, the treatment when a patient first has symptoms of a flu involves having the patient inhale the factor(s) that signal cells to reduce protein production. The factor(s) are now more quickly spread to different parts of the lung and more cells are signaled to reduce protein production. On an organ level, the factor(s) have warned a large area of epithelial cells to create an environment that decreases protein production. Why should we wait for viral propagation before signaling other cells? Facilitation of the signaling mechanism of cells by inhaling a mist of this factor(s) is analogous to an early warning system. On a cellular level, the inhalation of the factor has created a cellular environment that has diminished protein production (compared to normal) versus the typical rate of RNA destruction.

In the invention of this disclosure, factors signaling surrounding cells are a critical component in the success against a viral invasion. Surface cell receptors that respond to these factors and then decrease protein production are necessary. There are many such pathways and a few of the main ones are discussed here. Toll-like receptors (TLRs) play an important role in the immune system by recognizing pathogen-associated molecular patterns derived from various infectious agents. Some TLRs can recognize nucleic acids from infectious agents and can also recognize self-nucleic acids. TLR3 can recognize self-RNAs derived from damaged cells.

Another example of cell receptors that can recognize the factors of this disclosure are the pattern-recognition receptors (PRRs). The innate immune system employs germline-encoded PRRs for the initial detection of microbes. PRRs recognize microbe-specific molecular signatures known as pathogen-associated molecular patterns (PAMPs) and self-derived molecules derived from damaged cells, sometimes referred to in the literature as damage-associated molecules patterns (DAMPs). PRRs activate downstream signaling pathways that lead to the induction of innate immune responses by producing inflammatory cytokines, type I interferon (IFN), and other mediators. These processes trigger immediate host defensive responses such as inflammation but also initiate antigen-specific adaptive immune responses. Other PRRs include Toll-like receptors (TLRs), retinoic acid-inducing gene I- (RIG-I-) like receptors (RLRs), nucleotide-binding oligomerization domain- (NOD-) like receptors (NLRs), and C-type lectin receptors (CLRs). All the various PRRs included here and all their known agonists are included herein by reference.

The innate immune system does not have “memory” in the sense we understand that specific antibodies have memory. The “memory” that the innate immune system has is much more impressive. The contention that the immune system needs some kind of work-out or “priming” to be more effective only applies to the adaptive immune system and specific antibody generation. The innate immune system operates on fundamental factors that are different from SELF and NON-SELF. The adaptive immune response (antibodies) sense subtle differences in pathogens and require “priming”. The innate immune system senses marked differences in SELF from NON-SELF and this is built-in and doesn't have to be re-learned in every person as evidenced by the built-in PRR (pattern recognition receptors) that are activated by PAMPs (pathogen associated molecular patterns).

The human immune system evolved to defend against pathogens that are also constantly changing. A very important ability of the immune system is the ability to distinguish self from non-self. Evolution is not perfect. There will always be grey areas. This distinction between self and non-self is not perfect and auto-immune disorders in certain individuals is evidence of this. Just as a pharmaceutical company will try to find chemicals that hinder or damage bacterial life processes without affecting human life functions, the human immune system also functions this way. The immune system must first be able to detect structural features of the pathogen that is consistently distinct from the host. Once the immune system detects a molecular pattern that is shared by pathogens but not present in the human, it can activate a variety of mechanisms to inactivate the threat. The immune system has many effector mechanisms that have the ability to eliminate a large range of pathogen threats. In the lung, the innate immune response starts with the recognition of pathogen-associated molecular patterns by pattern recognition receptors (PRRs). Toll-like receptors are also expressed on many of the lung cells and are important PRRs. Once the PRRs are activated, further downstream signaling results in expression of interferons (IFNs) and other pro-inflammatory cytokines. Often a second round of signaling ensures that both infected and surrounding uninfected cells express a multitude of interferon stimulated genes (ISGs) that establishes an antiviral state.

Signaling mechanisms via PRRs lead to interferon production through MyD88-dependent (myeloid differentiation factor 88) and/or TRIF-dependent pathways via activation of essential transcription factors NF-κB and IRF (IFN regulator factors). These immune responses contribute to various pathogen clearance, including bacteria and viruses. However, the portion of the immune response that is more beneficial against bacteria pathogens is more generally cell-mediated and that portion of the activated immune response can be harmful to the host, but not necessary for viral clearance. A very useful aspect of the host immune response against viruses is the production of interferon which signals cells to decrease protein production. In this invention, since a PRR agonist is implemented to initiate the signaling of cells, the host immune response will often include a white blood cell mediated response which is not necessary but often more harmful. Decreasing white blood cell movement and activation is an important aspect of this invention. This is further elaborated upon in the section entitled “DECREASING WHITE BLOOD CELL MOVEMENT”.

The immune system can be separated into an innate immune system and an adaptive immune system. The innate immune system includes physical barriers such as epithelial cell layers and their tight junctions. The innate response also includes cytokines. The innate immune system also includes surface receptors and cytoplasmic proteins that bind molecular patterns expressed on the surface of invading pathogens. The lung epithelial cells are the largest surface in the human body that is in contact with the external environment. Every day the human respiratory tract is faced with 10,000 liters of inhaled air containing potentially airborne pathogens. The innate immune system response is much quicker than the adaptive immune system response. Antibody production can take up to two weeks from good exposure. By two weeks, a pathogen associated infection in the lungs can devastate the lungs. The innate immune system response is typically only mentioned in passing when referring to viral infections of the lung, but this author is convinced that the innate immune system response is by far the most important response to a pulmonary viral threat. The innate immune system also includes a cellular component comprised of macrophages/monocytes, granulocytes, neutrophils, natural killer cells, and dendritic cells. The cellular component of the innate immune system is much more useful against bacteria and yeast, but unlikely to be as important against viral respiratory infections.

The innate immune system has long been separated from the adaptive immune system by the innate system's lack of memory. This purely depends on how memory is described. On the scale of the individual, it can definitely be argued that the innate immune system appears not to have memory, although even this has been challenged since IFNs (interferon) can reprogram cell responses by leaving epi-genomic traces and effect future changes by post-translational modifications. This “memory” mechanism may endure for days to weeks. But on the scale of the human gene pool and on a multi-generational level, the pattern recognition receptors of the innate immune system can be considered “memory” since distinct structural components of bacterial cell walls such as LPS (lipo-polysaccharide) are recognized by cell receptors without that particular individual having previous exposure to the LPS since this LPS has been experienced over millions of years by our ancestors and this information is now hard-wired into cell receptors.

Typically, innate immunity includes various measures that inhibit or limit pathogen growth but generally thought to have little specificity and does not adapt during a subsequent infection with the same pathogen or does not generate long-lasting memory. The human innate immune system defenses include defensins, the complement system, white blood cells, and cytokines including interferons. Interferons have massive direct and indirect antiviral effects and many of these pathways have been well documented. These cytokines have direct antiviral effects and they also initiate subsequent adaptive immune responses. Viruses have also evolved strategies to antagonize the interferon response. Interferons can be induced by viral infections or even by antigenic stimuli.

The invention of this disclosure involves using a factor (or ligand, or pathogen-associated molecular patterns, commonly known as PAMPs) to activate pattern-recognition receptors (PRRs) that result in production of many pro-inflammatory molecules including interferons that ultimately result in protein kinase R phosphorylating eIF2α and less global protein synthesis which also reduces viral propagation. Timing of administration of this factor is critical. For example, the interferon response is tightly controlled and shortly after exposure to interferons, cultured cells enter an interferon-desensitized state that can last up to several days. Clearly, administration of this factor when a patient is NOT sick is not beneficial and the appropriate response to the factor may be LESS effective if the factor is administered chronically. The factor is ideally administered to the patient when the patient is confirmed to have a viral respiratory illness or when there is a moderate to high chance that the patient has been exposed to the virus and will develop a viral respiratory illness. The factor will be much more effective in the early stages of a respiratory viral illness. Later in the course of the viral illness, it is unlikely for the factor to be useful and may be more harmful (similar to interferon therapy for respiratory viral illnesses).

There are many published papers documenting the usefulness of interferons in treating viral infections. However, interferon therapy is expensive and it is unlikely that large scale use will be cost-efficient for respiratory viral infections that often have a low mortality rate. Also, interferon therapy is likely to be useful early on since it is the opinion of this author that the main benefit of interferon signaling is the ability to warn cells prior to being infected with a virus and once a cell is signaled by interferon, the cell can pro-actively relatively decrease protein production, thereby being better positioned to fight off a virus infection once it occurs. But, since very few patients who develop a respiratory viral infection are likely to pay for an expensive treatment at an early stage, the cost/benefit analysis is not favorable. The invention of this disclosure is to provide a factor that dramatically increases production of interferon at a much lower cost than interferon so that patients who have a respiratory viral infection are still likely to acquire the therapeutic and use it at an early phase of their viral infection. The therapeutic will jump-start interferon production and the interferon produced will then activate more production of interferon in an autocrine and paracrine fashion. Interferon also results in cells decreasing protein production and decreasing protein production creates a less hospitable cell environment for virus propagation.

Interferons in general are important in defending against viruses. In gene knockout studies in mice, it has also been revealed that type III IFNs play a crucial part in antiviral defenses of the respiratory tract epithelial surface. Among the IFN-induced proteins implicated in the antiviral actions of IFNs in virus-infected cells are PKR, OAS, and RNase L, the RNA-specific adenosine deaminase (ADAR), and the Mx protein GTPases. Interferon production mainly occurs in response to viruses and bacteria. Binding of molecules unique to pathogens such as viral RNA and bacterial endotoxin (LPS) by PRRs (pattern recognition receptors), also initiates release of IFNs. Other cytokines such as interleukins can also stimulate interferon production. Downstream, IFNs activate signal transducer and activator of transcription complexes (STAT). STAT activation initiates cell signaling pathways such as the Janus kinase-STAT (JAK-STAT) signaling pathway. Following activation, PKR catalyzes the intermolecular phosphorylation of several protein substrates including: the PKR protein itself, the a subunit of protein synthesis initiation factor 2, eIF-2α, and the transcription factor inhibitor IκB. Interferon-induced proteins affecting virus multiplication include PKR Kinase, which inhibits translation through the phosphorylation of eIF-2α and the OAS synthetase family and RNase L nuclease, which mediate RNA degradation. Well-studied anti-viral genes induced by type I interferons include protein kinase R (PKR), ADAR (adenosine deaminase acting on RNA), OAS, RNase L, and Mx proteins. PKR and ADAR lead to inhibition of translation initiation and RNA editing, respectively. OAS is a dsRNA-dependent synthetase that activates RNase L to degrade ssRNA.

IFNβ is initially activated by signals that then induce binding of several transcription factors (c-Jun/ATF-2, NF-κB, and interferon regulatory factor-3 (IRF3)), to the IFNβ promoter. NF-κB is activated my many different ligands to various PRRs and result in transcription of large classes of genes. However, IRF3 primarily regulates IFNβ production. IRF3 exists in a latent state in the cytoplasm of unstimulated cells but stimulation of the cell results in phosphorylation of IRF3 (either by serine-threonine kinases TANK-binding kinase-1 (TBK1/T2K/NAK) or the inducible IκB kinase (IKK-i/IKKε). IFNβ then dimerizes, moves into the nucleus, and binds to the coactivator CBP/P300 to activate production of IFNβ.

Interferon binds to a heterodimeric transmembrane receptor, the IFN-α receptor (IFNAR) and initiates a positive feed-back loop in an autocrine and paracrine manner. IFN binding to IFNAR activates Janus kinase 1 (JAK1) and tyrosine kinase 2 (Tyk2). JAK1 and Tyk2 in turn phosphorylate two transcription factors, signal transducer (STAT1) and activator of transcription 1 (STAT2). The phosphorylated STAT1 and STAT2 dimerize and together IFN-regulatory factor 9 (IRF9) form a transcription factor complex named IFN-stimulated gene factor 3 (ISGF3). ISGF3 translocates into the nucleus and binds to the DNA consensus sequence named IFN-stimulated response elements (ISRE), to induce expression of ISGs, a large group of “IFN-inducible” genes (over 300) including IRF7 and many others. IRF7, very similar to IRF3, is transcriptionally induced in many cell types as a target of type I IFN signaling. IRF7 also becomes activated by phosphorylation by TBK1 and/or IKK-1 and then activates further type I IFN expression.

When discussing the qualities or characteristics of these factors, it can be necessary to understand the state of the cell and surrounding cells. In a healthy cell state with little stress, the ratio of factor (or factors) present intracellularly versus extracellular is very high, in the local area of the cell and surrounding cells. This ratio can be 98, 99, 99.9, 99.99, or 99.999 percent in a healthy state. In an unhealthy cell state with stress in that local area of cells, the factor present extracellularly can increase many fold. Clearly, in a state when a formulation of factor is prepared for the patient and inhaled into the lung, this factor will be present in very high concentration (relative to normal concentration) extracellularly, in that local area. Also, when “factor” or “factors” are discussed, it is understood to be singular or plural and a combination of different categories of molecules or one category of molecule. All the different categories of molecules have the basic quality of interacting with a surface lung cell receptor that leads to phosphorylation downstream of the a subunit of eukaryotic translation initiation factor 2 (eIF2α) so that protein synthesis within the cell is relatively diminished. There are similar pathways to decrease protein synthesis by altering other initiation factors, which occur also during the stress response. All the categories of molecules will also have the at least 1, 2, 3, 4, 5, 6, 7 or 8 additional characteristics of factors discussed throughout this disclosure. Also, when discussing the different categories of molecules, aside from the primary quality of the factor, other qualities of the factors discussed may be specific for only that category of molecule.

Some categories of factors are included here and the sub-categories/variants are understood to be included. Well-known surface cell receptors of all factors listed are to be understood as included in this disclosure although not specifically mentioned. Categories of molecules (some are subcategories) that are effective factors include RNA, DNA, Damage-associated molecular patterns (DAMPs), Pathogen-associated molecular pattern (PAMPs), human RNA, human DNA, purine metabolites, uric acid, calreticulin, heat shock proteins, High Mobility Group Box 1 (HMGB1), HMGB1 variants, cell nuclear proteins (e.g. SAP130), bacterial/viral/yeast components, S100 proteins, well-known agonists of all immune receptors discussed in this disclosure, and well-known agonists of immune receptors leading downstream to phosphorylation downstream of a subunit of eukaryotic translation initiation factor 2 (eIF2α).

Many adjuvants have overlapping activity with PRR agonists and some of the factors mentioned above. Adjuvants that activate the innate immune system and increase interferon production are also understood to be included. Adjuvants were first described as “substances used in combination with a specific antigen that produced a more robust immune response than the antigen alone” (Ramon, 1924). The generally understood use of adjuvants is to increase the adaptive immune response (antibodies) to an antigen that is also given at around the same time as the adjuvant. The use of adjuvants in this disclosure is to provide a PRR agonist that activates the innate immune system and increases interferon production early in the clinical course of a viral infection. The purpose is not to increase antibody formation to a specific antigen that is also administered. It is also well understood that since adjuvants increase the immune response to antigens, adding a corticosteroid or other similar medication to decrease the immune response is not administered to the patient at a similar time. In this disclosure, the adjuvant or PRR agonist or COFs is combined with a therapeutic that decreases white blood cell migration and subsequent tissue damage associated with white blood cell activation. The use of the adjuvant in this disclosure is to activate PRRs so that the innate immune system is activated and processes within cells that are anti-viral are activated. The innate immune system includes other white blood cells that migrate and release destructive enzymes and proteases (such as macrophages, Killer T-cells, and neutrophils) but the goal of the therapeutic is to activate those cells as minimally as possible while still jump-starting the production of interferon so that sufficiently many cells are signaled early before the virus propagates. Adjuvants that do not activate the innate immune system are not included in the therapeutic of this invention. A general reference discussing adjuvants can be found in “The Theory and Practical Application of Adjuvants (D.E.S. Stewart-Tull ed. John Wiley & Sons 1995) and the information therein is incorporated by reference.

Corticosteroids (and analogues) are also discussed under the sections describing the 2nd and 4th fundamental strategies within this disclosure. In brief, cortisol is a catabolic hormone which effects decrease in protein production within cells. The advantages of decreased protein production in fighting a viral infection has been described in detail many times in this disclosure. Cortisol secretion within the human body has a diurnal pattern and is greater in the morning and less at night. More efficient allocation of available energy during activity improves survival value. If more of the food energy is converted into glucose and provides more fuel/energy for human activity (as opposed to using a greater portion of the food energy for cell growth), the individual has more energy necessary for all those tasks that require muscle activation and movement during the day. Cortisol's effects are more dominant during activity (typically day time) and growth hormone effects are more dominant during rest (night time). Cortisol is relatively more of a catabolic hormone and growth hormone is more of an anabolic hormone. Cortisol is a much smaller molecule than most cytokines and other peptide signaling molecules such as interferons and interleukins. It is the applicant's insight that once there is a virus infection, the body also uses the natural signaling molecules that result in decreased protein production such as cortisol. A system to transition from protein synthesis/cell growth (during states of inactivity, typically night time) to energy use and activity requiring muscle use (day time) is clearly in place and during a viral infection, partially adopting the portion of the system that decreases protein synthesis would be evolutionarily efficient; it is the applicant's insight that the body's signaling method to transition to energy use/less protein production (cortisol) is also adopted during virus infections. As previously stated, cortisol is a much smaller molecule than most cytokines and passing through barriers such as the blood brain barrier and the blood lung barrier. Cortisol is less than 400 Daltons in weight whereas human gamma interferon has a weight between 40,000 and 70,000 Daltons. Most land-based life forms on earth evolved in the presence of a clear diurnal cycle and most organisms were more active during daylight (since light facilitates movement) and these organisms for the same reason these organisms moved much less during night-time. Because of these needs, those organisms that evolved a system to allocate the available energy resources (food, etc.) more appropriately for the diurnal cycle of more/less activity would have a significant survival advantage. Using relatively more energy resources to “grow” cells versus using the available energy resources for fuel to enable activity would be a survival disadvantage. Growth hormone signals cells to “grow” during periods of inactivity (night time) and cortisol signals cells to “grow less” to provide more energy resources for muscles (during the day). This signaling must be done quickly and the response must be within the time frame of minutes to hours to have benefit. It just so happens that limiting protein synthesis is also an incredibly useful strategy in the fight against viruses. Why would any organism not use this signaling system that diverts resources to muscles/limits protein synthesis, when this signaling system has been so well organized to reach almost all cells within an organism? Typically, cortisol is considered a “stress hormone”. It would be more aptly termed an “activity hormone”. Cortisol is considered an “anti-inflammatory” hormone. It is much more accurate to consider it an “anti-virus” hormone. Cortisol was probably the very first “anti-viral” signaling molecule, evolutionarily speaking. Cortisol is a much smaller molecule than interferon. Larger molecules typically take longer to evolve than smaller molecules. Genomic analysis has revealed that up to 30% of human genes have a glucocorticoid response element and the large percentage of genes that have this element show how ancient cortisol is and how pervasive its affect.

Here is an example of how cortisol can function to produce an anti-viral environment in cells. A virus infection occurs. The innate immune system is activated and cytokines are generated. Cytokines can activate the hypothalamus-pituitary axis (HPA) which produces cortisol. Pro-inflammatory cytokines that produce fever can also dislodge significant amounts of glucocorticoids from corticosteroid binding globulin (CBG) and free cortisol is biologically active and more available to have an effect on target cells. Cortisol is a small molecule and is carried by the blood stream to all parts of the body and can even pass through the blood brain barrier. These features make the cortisol molecule a very efficient signaling molecule. Once cortisol is within cells, it acts to decrease protein synthesis. Less virus RNA/DNA is converted into virus protein parts and viral replication is slowed. Ribonucleases have more time to chop up virus RNA. There is not a COVID patient in the world that has healed without the benefit of ribonucleases; yet, ribonucleases are relatively unknown and most people on earth are aware of COVID neutralizing antibodies. Ribonucleases are the least appreciated molecules on earth.

### Specific Factor Discussion

In another embodiment of the present invention, a mixture of a factor or molecule discussed in this disclosure under the first fundamental strategy section can be combined with any factor or molecule discussed under the second and/or third fundamental strategy sections; the resultant mixture can be combined with any molecule discussed under the section describing decreasing white blood cell movement (fourth fundamental strategy). In accordance with a specific embodiment of the present invention, a mixture comprising a molecule discussed in the third fundamental strategy can be combined with a factor discussed in the first and/or second fundamental strategy sections; this resultant mixture can also be combined with any molecule discussed under the section describing decreasing white blood cell movement.

In another embodiment, the medication provided to the patient is selected from any of the discussed Four Fundamental Strategies.

In another embodiment, the medication provided to the patient is a combination of substances or factors from at least two of the discussed Four Fundamental Strategies.

In yet another embodiment, the medication provided to the patient is a combination of substances or factors from at least three of the discussed Four Fundamental Strategies.

In yet another embodiment, the medication provided to the patient is a combination of substances or factors from all four of the discussed Four Fundamental Strategies.

In yet another embodiment, the patient is infected with a DNA virus.

In yet another embodiment, the patient is infected with an RNA virus.

An objective of the disclosure of this invention is to provide a factor in an inhaler to be inhaled around the time of early flu-like or cold symptoms to cause on a molecular level, signaling to lung cells that results in overall decreased protein production in those signaled cells, lasting for a duration of several days to weeks. An aerosol compound comprising at least two separate categories of molecules (chosen from within the list of factors able to signal cell receptors initiating the stress response) but including from 2 separate categories of molecules up to 100 separate categories of molecules. The benefit from having different categories of molecules will be explained, but a single factor may also be used. In an example of a final prepared compound (FPC) having 5 different categories of factors, each comprising 20% of the prepared compound for human inhalation, each of the 5 different molecules have the essential characteristic of being able to interact with a surface cell receptor followed by phosphorylation downstream of a subunit of eukaryotic translation initiation factor 2 (eIF2α) so that protein synthesis within the cell is relatively diminished. Yet each category will have a different side effect profile. Each category will have other effects aside from the essential characteristic. So having a compound with 5 different categories distributes the side effect profile of the prepared compound, with less possibility of damage. For example, if one of the 5 different categories has a mild side effect on the liver, the body will much more easily handle a final prepared compound having only 20% of the liver affecting category versus formulating the final prepared compound with only the factor that has a mild side effect on the liver. The advantage of distributing the side effect profile by increasing the number of different categories of molecules for the final prepared compound, gradually diminishes after about 100 categories. If a final prepared compound has a 100 categories of factors comprising the whole and if each category is allotted about 1% by weight of the final prepared compound, then it can be seen that the side effect profile of that one category is only about 1% effect of what the side effect profile would be if the final prepared compound is comprised of that one category completely, wherein the side effect profile would then be 100% (relative to when the category is only 1% by weight in the final prepared compound). When the FPC is comprised of 2 categories, one category can comprise in percent by weight identity, any fractional percentage, in intervals of 0.01%, between 50.00% and 95.00% of the FPC.

If the FPC is comprised of 3 categories, any of the 3 categories can comprise in percent by weight identity, any fractional percentage, in intervals of 0.01%, between 33.33% and 95.00% of the FPC. The first percentage in the range (33.33% to 95.00%) is derived by taking 100% divided by the number of categories which in this case is 3. The other 2 categories must share, 100% minus the first selected category percentage weight. If the first category in this case is 33.33%, the other 2 categories must share 66.67% and the 66.67% is now assumed to be 100% and we can use the method described earlier for 2 categories, in determining the relative weight percentage.

If the FPC is comprised of 4 categories, one category can comprise in percent by weight identity, any fractional percentage, in intervals of 0.01%, between 25.00% and 95.00% of the FPC. The other 3 categories must share, 100% minus the first selected category percentage weight. If the first category in this case is 25.00%, the other 3 categories must share 75.00% and the 75.00% is assumed to now be 100% and we can use the method described in the previous paragraph for 3 categories, in determining the relative weight percentage of the remaining 3 categories.

This is continued in this fashion up to 100 categories wherein one category can comprise in percent by weight identity, any fractional percentage, in intervals of 0.01%, between 1.00% and 95.00% of the FPC. Later in this disclosure, this method of creating varied compositions will be used also DNA and/or RNA as 100% of the FPC and DNA and/or RNA as one category of the FPC.

As discussed previously, many of these factors activate the innate immune system which also includes increased white blood cell migration, movement, and release of proteolytic and destructive enzymes from these white blood cells. Corticosteroids and similar compounds can be added to the FPC. When the innate immune system is strongly activated and when activation of white blood cell movement and proteolytic enzyme release is destructive or expected to be significant, corticosteroids can be administered a day prior to administering the FPC to the patient (or within 24 hours before or after administering the FPC) or corticosteroids can be administered as one of the components of the FPC. Many similar therapeutics are available that limit white blood cell movement and limit white blood cell release of proteolytic enzymes while still permitting the factor or factors to decrease protein synthesis within a cell. The goal of administering the corticosteroid is to limit white blood cell movement and release of proteolytic enzymes. Depending on the dosage of aerosolized factor administered, no corticosteroid may be needed (if very low dosage of factor administered), aerosolized corticosteroid may be needed (some inhibition of white blood cell movement) or aerosolized corticosteroid with an oral dose of corticosteroid may be required. The dosage of corticosteroid may be repeated as necessary.

Of all the factors discussed above, DNA and RNA are by far the most varied in composition and require a more in depth discussion. Regarding mRNA vaccines, a few very well-funded companies are producing mRNA vaccines that are currently in trials. Efficient in vivo mRNA delivery is critical to achieving therapeutic relevance for these companies since the goal is to deliver the exogenous mRNA into the cell cytoplasm to be translated into protein for those companies. For companies who use RNA, those companies fall under several broad areas but they all require the RNA that they use to have a certain code. If the code varies compared to their desired code by more than 10% or more, it is unlikely to have the desired therapeutic result. If the RNA does not reach the cytoplasm, it will not be translated into protein and unlikely to have the desired therapeutic result. We will lump these companies under “Gene Therapy Companies” (GTC) and we will constantly refer back to this to show how the DNA and RNA of this disclosure is drastically different both in terms of structure and ultimate function. The GTC goals are to actually produce protein and to have the delivered mRNA translated into protein. Various vectors have been used. When mRNA is injected into the body, it can trigger virus-detecting immune sensors. That causes cells to shut down protein production, preventing the very therapy that the mRNA is trying to accomplish. The goal of mRNA is to prompt cells to make proteins. A breakthrough in converting mRNA into useful therapeutics was from a simple discovery, replacing uridine in RNA with pseudo-uridine. This modified mRNA evaded immune sensors. You can see the therapeutic mRNA industry desires to make specific proteins with their inserted mRNA and not trigger immune responses. GTC uses pseudo-uridine in their mRNA to reduce immune responses. In the “invention of this disclosure” (IOTD), uridine is more useful. GTC favor 5′mRNA cap modification to improve translation. IOTD, a reverse cap or no cap is more useful since it hinders translation. Unlikely for IOTD RNA to enter cell cytoplasm since no mRNA delivery co-formulation or vector for intracellular delivery of nucleic acid is provided, since the goal for the RNA as a factor is to activate the stress response via surface cell receptors on lung cells. GTC's engineer their mRNA to display low immunogenicity, prolonged stability, and potent translation, along with a suitable vector. IOTD, the RNA is more adequately described as average or higher immunogenicity, average or lack of stability, and average to no translation, and no vector or other delivery system into the cell.

GTC have a specific goal for their mRNA and deviation in nucleotide sequence by (in intervals of 0.01%) by 3% up to 50% is likely to significantly reduce its intended function and its intended biological activity. For IOTD, referring to the RNA and or DNA nucleotide sequence, deviations from all known, used, published DNA and/or RNA sequences, (in intervals of 0.01%) from 3% to 100% deviation in nucleotide sequence. For IOTD, in various embodiments, nucleic acid sequences have been sufficiently modified so that compared to the original sequence (which either on its own or from protein translation has its understood biological activity), there is substantially comparatively little or minimal biological activity relative to the native original code. For GTC, preparation of the mRNA and then providing a suitable vehicle for delivery to the cytoplasm of the cell is far from inexpensive and the author is not aware of any prior art describing the preparation of RNA with a random sequence for delivery to the lung cells only to stimulate the lung cells stress response. For IOTD, that is one of our objectives, to deliver RNA that has a sequence which if translated into a protein, does not have significant biological activity. For GTC, for a given mRNA treatment to a patient, most of the mRNA molecules in that one treatment are similar or identical to one another and it will be understood that the practitioner desires all the mRNA molecules to have substantially the same biological function, which is determined by the nucleotide code similarity.

For IOTD, in another embodiment, a FPC is comprised of RNA or DNA for at least one of its categories. RNA and or DNA is so varied, that we will name any RNA strand that is different by (in intervals of 0.01%) 10% to 100% in size and or code, a variant. The nucleic acid molecule may contain at least about 5, 10, 15, 20, 25, 30, 35, 40, 45, 100, 150, 200, 250, 300, 350, 400, 450, 500, 550, 600, 650, 700, 750, 800, 850, 900, 950, 1000, 1050, 1100, 1150, 1200, 1250, 1300, 1350, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, or more contiguous nucleic acids in length.

When the FPC is comprised of 2 variants, one variant can comprise in percent by weight identity, any fractional percentage, in intervals of 0.01%, between 50.00% and 99.90% of the FPC. The other variant would be determined by,

If the FPC is comprised of 3 variants, any of the 3 variants can comprise in percent by weight identity, any fractional percentage, in intervals of 0.01%, between 33.33% and 95.00% of the FPC. The first percentage in the range (33.33% to 95.00%) is derived by taking 100% divided by the number of variants which in this case is 3. The other 2 variants must share, 100% minus the first selected variant percentage weight. If the first variant in this case is 33.33%, the other 2 variants must share 66.67% and the 66.67% is now assumed to be 100% and we can use the method described earlier for 2 variants, in determining the relative weight percentage.

If the FPC is comprised of 4 variants, one variant can comprise in percent by weight identity, any fractional percentage, in intervals of 0.01%, between 25.00% and 95.00% of the FPC. The other 3 variants must share, 100% minus the first selected variant percentage weight. If the first variant in this case is 25.00%, the other 3 variants must share 75.00% and the 75.00% is assumed to now be 100% and we can use the method described in the previous paragraph for 3 variants, in determining the relative weight percentage of the remaining 3 variants.

This is continued in this fashion up to 10,000 variants wherein one variant can comprise in percent by weight identity, any fractional percentage, in intervals of 0.01%, between 0.01% and 99.90% of the FPC.

### Specific Factor Discussion

LPS is an example of a factor or ligand that activates a PRR. Inhaled LPS has been used to determine the competence of the innate immune system. Inhaled LPS has also been used in animal studies to support the hypothesis that bacterial LPS is important in the pulmonary reaction induced by organic dusts. It has not been used as an inhaled therapeutic for a viral respiratory illness. LPS has also been used to show the benefits of LPS antagonist drugs in preventing the development of environmental airway disease. There is much more literature documenting the harmful effects of LPS on lung tissue. A micro-dose of aerosolized LPS used as a signaling factor at the onset of a respiratory viral illness is a new concept. As an analogy, botulinum toxin at a large dose can paralyze and even kill, but at a micro-dose of botulinum toxin, a reduction of skin wrinkles is widely used and is beneficial. A micro-dose of aerosolized LPS at the onset of a respiratory viral illness will effectively signal many alveolar epithelial cells to prepare for a spreading respiratory viral infection and lead to decreased symptoms. Even water at large doses is toxic to the human. Many cells in the human body have PRRs for LPS. That is what the pattern recognition receptor is there to do, detect pathogen ligands such as LPS. Just as a vaccine provides a portion of the pathogen and the body creates antibodies to the vaccine but the vaccine molecule is unable to replicate on its own and yet the advantages of the vaccine molecule and the resulting antibody that is created is apparent; the LPS is a portion of the pathogen that cannot replicate and there are PRRs that detect the LPS and respond accordingly and the cells benefit from the LPS signaling and can produce interferons and other inflammatory molecules that create an “anti-viral state” in the cell and the cell benefits. This discussion applies to all ligands for PRRs.

LPS activates TLR-4 which leads to TLR4-induced activation of the transcription factor nuclear factor kappa B (NF-κB). NF-κB regulates expression of such pro-inflammatory molecules such as interferons. TLR4 can also signal via the TRIF-dependent pathway after endocytosis and trafficking to the endosome, where it recruits TRIF through TRAM. The TLR4-TRAM-TRIF branch also activates NF-κB and also IRF3 activation with subsequent type I interferon production. Interferons activate protein kinase R which phosphorylates eIF2α causing diminished viral protein translation. However, LPS also activates the cell mediated response to pathogens and the recruitment of white blood cells and the subsequent release of proteolytic enzymes can damage tissues. Inhalation of aerosolized LPS activates local production of interferons. It is well known that alveolar epithelial cells express functional TLR-2 and TLR-4. If a systemic corticosteroid or other immunosuppressant is present in the body, white blood cell movement is drastically reduced. The relative decrease in white blood cell movement and subsequent release of proteolytic enzymes by the white blood cells is a necessary component to activating the innate immune response. The anti-viral response to the LPS is generally preserved but the cell mediated response to the LPS is mostly inhibited.

The present invention also encompasses LPS (lipopolysaccharide) or lipid A variants, derivatives, and analogs. The derivatives and analogs of factors discussed under the first through third strategy may also be used in various embodiments of this invention.

It is well known that interferons reduce viral replication both in vitro and in vivo. There are many companies that are advocating various interferons in treating COVID-19. However, interferon therapy is expensive and many researchers have noted that interferon therapy is much less useful in later more severe COVID-19 cases. Interferons are part of the signaling system to warn uninfected cells to decrease by inducing the release of intracellular enzymes including 2 ′5′ oligoadenylate synthetase and protein kinase R (double-stranded RNA dependent protein kinase), interferons inhibit protein synthesis and slow down viral replication. However, since many early mild cases of COVID-19 are not likely to progress to severe cases, a much less expensive treatment is necessary for mild cases. The use of DAMP and PAMP molecules to activate PRRs (pattern recognition receptors) which activates expression of various pro-inflammatory molecules including interferons is a much less expensive solution for early, mild cases of respiratory viral illnesses. Many of these PRRs, when activated, increase the activity of protein kinase R and reduction of protein synthesis creates an anti-viral state. There are many natural and synthetic agonists of these PRRs and PRRDB 2.0 (pattern recognition receptor data bank, DATABASE, The Journal of Biological Databases and Curation) is an updated version of PRRDB and this data bank has 2700 entries of PRRs and their ligands. This database also includes references to 4500 experimentally determined structures in the protein data bank of various PRRs and their ligands and these are all incorporated herein in their entirety by reference.

Nonlimiting examples of PRR agonists that can be used in the compositions and methods of this invention include DAMP molecules (mitochondria, granules, endoplasmic reticulum, the plasma membrane), Biglycan, Decorin, Versican, hyaluronan, Heparan sulfate, Fibronectin, Fibrinogen, Tenascin C, Uric acid, S100 proteins, Heat-shock proteins (including HSP70, HSP90), ATP (including adenosine and other nucleotides), purine metabolites, F-actin, Cyclophilin A, Histones, HMBG1 (High mobility group box 1), HMGN1, SAP130, DNA, RNA, mitochondrial DNA, mitochondrial RNA, Mitochondrial ROS, Calreticulin, Defensins, Cathelicidin, Eosinophil-derived neurotoxin, Granulysin, Syndecans, Glypicans, RIG-like receptor agonists (including RIG-1, MDAS, and LGP2), PAMPS, LPS, bacterial lipoproteins and lipopeptides, peptidoglycan from gram positive bacteria cell wall, porins from the outer membrane of Gram-negative bacteria cell wall, lipoteichoic acid, lipoarabinomannan, mycolic acid, mannose-rich glycans, flagellin found in bacterial flagella, bacterial and viral nucleic acid, DNA with high CpG sequences, N-formylmethionine, double-stranded viral RNA, zymosan from yeast cell walls, fungal glucans, chitin, phosphorylcholine and other lipids common to microbial membranes, C-type lectin receptor (CLRs) agonists, NOD-like receptor agonists, RIG-I-like receptor agonists, TLR2 ligands (including heat killed bacteria, cell wall components, synthetic lipoproteins, Pam3CSK4), TLR 3 ligands (including Poly (I;C), dsRNA), TLR4 ligands (including LPS and variants), TLR5 ligands (including flagellin), TLR7/8 ligands (including single stranded RNAs and also small synthetic molecules such as imidazoquinolines and nucleoside analogs), TLR9 ligands (including unmethylated CpG motifs), NOD1/NOD2 agonists (such as peptidoglycan and muramyl dipeptide structure), RLR ligands (5′ppp-dsRNA, 3p-hpRNA, Poly (I:C), CLR ligands (including curdlan, HKCA, laminarin, Pustulan, Scleroglucan, inflammasone inducers, ADP-Heptose, Dual TLR agonists (such as adilipoline) and RIG-1 like receptor (RLR) ligands.

Decreasing White Blood Cell Migration and/or Activation (the Fourth Fundamental Strategy)

As previously explained at length, detection of a virus infection activates a cascade of events. Signaling other cells of a virus invasion is critical in mitigating the damage from a virus infection. The invention of this disclosure involves facilitating the signaling process so that more cells are warned prior to being infected by a virus. In some embodiments of the invention of this disclosure, a signaling factor is used that can activate white blood cell movement and migration into the virus infected area. Using certain PRR agonists as a signaling agent will decrease protein production within cells (very useful in the fight against viruses) but can also activate white blood cell migration and/or activation and cause release of tissue damaging factors from those white blood cells. This is destructive and not necessary for the fight against virus infections. Adding a therapeutic to decrease white blood cell movement/migration can be necessary. Since PRR agonists can also result in recruitment of various immune cells (that can release tissue damaging molecules) to the site of the PRR agonist activity, a therapeutic to decrease recruitment of immune cells is also necessary when the dosage of PRR agonist(s) is sufficiently high to induce tissue damage. The use of PRR agonists initiate anti-viral activity (the three fundamental strategies described herein) but may also increase recruitment of polymorphonuclear neutrophils (PMN) and macrophages and the production of microbicidal agents which can also cause tissue damage. Recruitment of white blood cells is useful for clearance of bacterial infections but not nearly as effective against virus infections as the described three fundamental strategies against viruses. Furthermore, PMN and white blood cell recruitment and subsequent release of damaging molecules from these white blood cells can cause more tissue damage and may even kill the host. An objective of this invention is to relatively increase the three fundamental strategies described herein by use of PRR agonist but since PRR agonists also induce recruitment of white blood cells, adding a therapeutic to decrease the relative recruitment of white blood cells. Any of the therapeutics described under this “Fourth fundamental strategy” can be combined with factors described under the first three fundamental strategies. The therapeutic can be administered slightly before, at the same time, or slightly after the administration of the PRR agonist.

The applicant's view of the fourth fundamental strategy is in stark contrast to the current view of corticosteroids. Cortisol is considered an “anti-inflammatory”, as if cortisol inhibits the action of the immune system in its efforts against pathogens. As previously explained under the other “fundamental strategies”, cortisol is critical in allocating necessary energy resources into activity (muscle movement) by increasing gluconeogenesis and decreasing growth (protein synthesis). Since viruses do not have their own protein making machinery, viruses rely on the hosts cells to perform that function. If the host cells decrease RNA translation into protein and generally decrease protein synthesis, the cell creates a less hospitable environment for viruses to propagate. The diurnal variation in growth versus non-growth for host cells is mediated by hormones and growth hormone signals cells to grow and cortisol signals cells to limit growth so more energy is available for activity during the day. Decreasing protein synthesis due to cortisol is also an incredibly useful strategy against viruses since viruses rely on the host cell to produce virus protein parts. Cortisol is an ancient molecule and to understand it correctly one must understand its role in managing limited energy use and how the host organism adopted cortisol's signaling ability in the body's defense against virus infections.

Cortisol was probably the first signaling molecule in organisms in their battle against virus infections, due to its ability to signal cells quickly to reduce protein synthesis. The host strategies against bacterial infections are by nature very different. But, bacterial infections are also evolutionarily likely to use the “signaling system” used by the host in its fight against virus infections. As evolution of the immune system progressed and recruitment of white blood cells and release of their damaging substances were added, controlling the damage effected by the white blood cells against bacterial infections was necessary, since the early signaling systems basic framework against pathogens was shared by the body in its defense against viral or bacterial infections. Since probabilistically, virus detection was more difficult but a lack of a quick global host response to a viral infection was also more devastating for the host, a more robust host response to a virus infection was necessary and sometimes the host responded in spite of no virus infection (a false positive). The down-side to the host responding quickly and globally to a possible virus infection when there was no virus infection was not costly to the organism, aside from the few days of decreased growth. The cost to not responding quickly and globally to a virus infection was death. Now, since the bacterial infection signaling system also adopted the signaling system used by the host against viral infections, and since the host response against bacterial infections included the recruitment of white blood cells, migration of white blood cells to the site of infection, and release of tissue damaging substances from white blood cells, it was critical to raise the threshold for all aspects of white blood cell involvement against bacteria since the signaling system (used by both viruses and bacteria) evolved to over-call virus detection (since more difficult to detect viruses but lack of an early and global response against a viral infection might be catastrophic for the organism) and since bacterial infections were less likely to grow as quickly and less likely to spread as quickly. The host used the backbone signaling system for both virus and bacterial infection and that signaling system had to be quick and far-reaching for it to be effective against viruses. This same backbone signaling system was also adopted by the host in its defense against bacterial infections but the strategy to overcome bacterial infections did not need to be nearly as quick yet more locally damaging (white blood cell recruitment and subsequent release of damaging proteases/substances). Bacteria grow much more slowly than viruses and generally do not spread as quickly as viruses within a host. Bacterial infections are also more easily detected as most bacterial infections are not intracellular. But, the actual “weaponry” the host must use against bacteria is much more intense at the actual site of bacterial infection. The backbone signaling system uses a virus detection system that “overcalls” virus presence and the signaling system is quick and far-reaching. However, the effective strategy against viruses versus bacteria is very different. Any pathogen invasion requires the host to first detect the pathogen, then communicate that information to other cells, and finally to respond to the pathogen (quash, quell, destroy, eradicate) in an effective manner. Also, the benefits of communicating the presence of a pathogen are much greater for a virus infection than a bacterial infection. A relative decrease in protein synthesis within a cell creates a less hospitable environment for viral propagation and so warning a cell (signaling) in advance of an infection is tremendously useful. It is difficult to imagine a strategy that is pro-active and that useful when considering bacterial infections. Warning an “area of tissue” that there might be a bacterial infection coming may be useful because white blood cells can be recruited to the area and ready to discharge their damaging proteases, but since bacteria grow more slowly, the benefit of the “signaling” and the pro-active response would be much less. The benefits to having a “signaling” system to warn other cells is not nearly as useful for the host defense against bacterial infections as it is for viral infections. Accordingly, using the basic “signaling” mechanism for virus infections (e.g., cortisol) to also reduce white blood cell migration is beneficial for these reasons.

Many of the PRR agonists also upregulate the expression of PMN specific activator genes which can result in dramatic increase in PMN infiltration into the lung tissue. A goal of this fourth fundamental strategy is to provide a white blood cell migration minimizing medication such that the white blood cell migration and/or activation is about 20% less than use of PRR agonist alone.

In yet another embodiment, a white blood cell migration and/or activation minimizing medication is provided in conjunction with a PRR agonist such that the white blood cell migration and/or activation is about 30% less than use of PRR agonist alone.

In yet another embodiment, a white blood cell migration and/or activation minimizing medication is provided in conjunction with a PRR agonist such that the white blood cell migration and/or activation is about 40% less than use of PRR agonist alone.

In yet another embodiment, a white blood cell migration and/or activation minimizing medication is provided in conjunction with a PRR agonist such that the white blood cell migration and/or activation is about 50% less than use of PRR agonist alone.

In yet another embodiment, a white blood cell migration and/or activation minimizing medication is provided in conjunction with a PRR agonist such that the white blood cell migration and/or activation is about 60% less than use of PRR agonist alone.

In yet another embodiment, a white blood cell migration and/or activation minimizing medication is provided in conjunction with a PRR agonist such that the white blood cell migration and/or activation is about 70% to 100% less than use of PRR agonist alone.

In yet another embodiment, the composition provided to the patient is a PAMP molecule combined with a white blood cell migration/activation inhibitor.

In yet another embodiment, the composition provided to the patient is a DAMP molecule combined with a white blood cell migration/activation inhibitor.

In yet another embodiment, the composition provided to the patient is a TLR agonist combined with a white blood cell migration/activation inhibitor.

In yet another embodiment, the composition provided to the patient is a NOD-like receptor agonist combined with a white blood cell migration/activation inhibitor.

In yet another embodiment, the composition provided to the patient is a CLR agonist combined with a white blood cell migration/activation inhibitor.

In yet another embodiment, the composition provided to the patient is a RIG-1 like receptor agonist combined with a white blood cell migration/activation inhibitor.

The innate immune system includes white blood cells including monocytes/macrophages and neutrophils. The activation of the innate immune system can also result in recruitment of these various cells. However, these cells can result in release of very potent enzymes and proteases that may be very useful for bacteria but can potentially cause much more harm for the host. The primary defenses against viruses that eukaryotic cells have evolved are short term decreased protein production and ribonucleases. Viruses do not have protein making machinery and rely on their host to produce their proteins. They are highly evolved to enter cells and if that ability was not exceptional, it would be extremely unlikely for that particular virus to be able to propagate at a significant rate and become a pandemic. That also means that viruses are mostly within eukaryotic cells and that the amount of time these viruses spend outside of cells once in an organism is low. Evolutionarily speaking, it would be difficult to explain the utility of antibodies which are mainly extracellular in the fight against viruses which are going to mostly be intracellular. Both the innate immune system and the adaptive immune system mobilize immune cells. It is unlikely that white blood cells that release proteolytic enzymes that are highly non-specifically destructive will be useful in the human body's defense against viruses. Cytokines and other signaling protein production by these white blood cells may be useful when those cytokines result in an anti-viral state within cells that receive the signal. But, proteolytic and destructive enzymes released by these white blood cells are not useful. A non-specific molecule that activates the innate immune system and that results in phosphorylation of eIF2α will create an anti-viral environment within cells but that non-specific molecule may also activate white blood cells which then can destroy tissue. Immobilizing white blood cells and preventing white blood cells from releasing their destructive enzymes is absolutely necessary when using molecules that can activate the innate immune system. Glucocorticoids cause immunosuppression. The main mechanism is through inhibition of nuclear factor factor kappa-light-chain-enhancer of activated B cells (NF-κB). NF-κB is a necessary transcription factor involved in the synthesis of many mediators that promote the immune response and therefore inhibition of this factor diminishes the immune response. Glucocorticoids also suppress cell-mediated immunity by decreasing production of many cytokines, especially IL-2. The primary anti-inflammatory effect of glucocorticoids is Lipocortin-1 production which suppresses phospholipase A2, which blocks eicosanoid production and inhibits various white blood cell inflammatory events including chemotaxis and phagocytosis.

Many anti-inflammatory drugs reduce white blood cell migration, movement, activation, and release of destructive enzymes, while still allowing PRR receptor activation to produce interferon and other signaling cytokines, ultimately resulting in decrease cell protein synthesis. Glucocorticoid drugs include beclomethasone, betamethasone, cortisone, dexamethasone, hydrocortisone, methylprednisolone, prednisolone, prednisone, triamcinolone, flunisolide, fluticasone, and budesonide. Many other drugs used in auto-immune disorders and transplant patients also suppress white blood cell movement/migration while allowing PRR receptor activation resulting in decreasing cellular protein synthesis. Other drugs that are useful in reducing white blood cell movement and/or migration and/or activation include colchicine, ciclosporin, non-steroidal anti-inflammatory medications, azathioprine, chloroquine, cyclophosphamide, dapsone, everolimus, etanercept, fingolimod, hydroxychloroquine, lenalidomide, methotrexate, mycophenolate mofetil, pomalidomide, penicillamine, sirolimus, sulfasalazine, sodium aurothiomalate, tacrolimus, zotarlimus, anti-CD20 biologics (rituximab), Belimumab, Abatacept, anti-IL-17 biologics, (secukinumab), anti-IL-23 biologics (guselkumab), anti-IL-12/23 biologics (ustekinumab), anti-IL-5 biologics, decreasing lymphocyte movement (e.g., vedolizumab), cytostatics, and other biologics.

### Pharmaceutical Preparations and Methods of Administration

In some embodiments, the compositions described herein are formulated in a pharmaceutically acceptable carrier. Those compositions can be administered to a subject at therapeutically effective doses to treat any viral infection. The subject can be any mammal, reptile or avian, including horses, cows, dogs, cats, sheep, pigs, and chickens, and humans.

### Therapeutically Effective Dosage

Toxicity and therapeutic efficacy of such compositions can be determined by standard pharmaceutical procedures in cell cultures or experimental animals for determining the LD50 (the dose lethal to 50% of the population) and the ED50, (the dose therapeutically effective in 50% of the population). The dose ratio between toxic and therapeutic effects is the therapeutic index that can be expressed as the ratio LD50/ED50. Compositions that exhibit large therapeutic indices are preferred. While compositions exhibiting toxic side effects may be used, care should be taken to design a delivery system that targets such compositions to the site affected by the disease or disorder in order to minimize potential damage to unaffected cells and reduce side effects.

The data obtained from the cell culture assays and animal studies can be used in formulating a range of dosages for use in humans and other mammals. The dosage of such compositions lies preferably within a range of circulating plasma or other bodily fluid concentrations that include the ED50 with little or no toxicity. The dosage may vary within this range depending upon the dosage form employed and the route of administration utilized. For any composition of the invention, the therapeutically effective dose can be estimated initially from cell culture assays. A dosage may be formulated in animal models to achieve a circulating plasma concentration range that includes the EC50 (the concentration of the test composition that achieves a half-maximal effect) as determined in cell culture. Such information can be used to more accurately determine useful dosages in humans and other mammals. Composition levels in plasma may be measured, for example, by high performance liquid chromatography.

The amount of a composition that may be combined with pharmaceutically acceptable carriers to produce a single dosage form will vary depending upon the host treated and the particular mode of administration. It will be appreciated by those skilled in the art that the unit content of a composition contained in an individual dose of each dosage form need not in itself constitute a therapeutically effective amount, as the necessary therapeutically effective amount could be reached by administration of a number of individual doses. The selection of dosage depends upon the dosage form utilized and the viral infection being treated.

The dosage regime for treating a viral infection with the compositions and/or composition combinations of this invention is selected in accordance with a variety of factors, including the type, age, weight, sex, diet and medical condition of the patient, the route of administration, pharmacological considerations such as activity, efficacy, pharmacokinetic and toxicology profiles of the particular composition employed, whether a composition delivery system is utilized and whether the composition is administered as a pro-drug or part of a drug combination. Thus, the dosage regime actually employed may vary widely from subject to subject.

### Formulations and Use

The compositions of the present invention may be formulated by known methods for administration to a subject using several routes which include, but are not limited to, parenteral, oral, topical, intradermal, intramuscular, intraperitoneal, intravenous, subcutaneous, intranasal, epidural, inhaled and ophthalmic routes. The individual compositions may also be administered in combination with one or more additional compositions of the present invention and/or together with other biologically active or biologically inert agents (“composition combinations”). Such biologically active or inert agents may be in fluid or mechanical communication with the composition(s) or attached to the composition(s) by ionic, covalent, Van der Waals, hydrophobic, hydrophilic or other physical forces. It is preferred that administration is localized in a subject, but administration may also be systemic.

The compositions or composition combinations may be formulated by any conventional manner using one or more pharmaceutically acceptable carriers and/or excipients. Thus, the compositions and their pharmaceutically acceptable salts and solvates may be specifically formulated for administration, e.g., by parenteral, inhalation or insufflation (either through the mouth or the nose) or oral, buccal, parenteral or rectal administration. The composition or composition combinations may take the form of charged, neutral and/or other pharmaceutically acceptable salt forms. Examples of pharmaceutically acceptable carriers include, but are not limited to, those described in Remington's Pharmaceutical Sciences (A.R. Gennaro, Ed.), 20th edition, Williams & Wilkins PA, USA (2000).

The compositions may also take the form of solutions, suspensions, emulsions, tablets, pills, capsules, powders, controlled- or sustained-release formulations and the like. Such compositions will contain a therapeutically effective amount of the composition, preferably in purified form, together with a suitable amount of carrier so as to provide the form for proper administration to the patient. The formulation should suit the mode of administration.

### Parenteral Administration

The composition or composition combination may be formulated for parenteral administration by injection, e.g., by bolus injection or continuous infusion. Formulations for injection may be presented in unit dosage form in ampoules or in multi-dose containers with an optional preservative added. The parenteral preparation can be enclosed in ampoules, disposable syringes or multiple dose vials made of glass, plastic or the like. The composition may take such forms as suspensions, solutions or emulsions in oily or aqueous vehicles, and may contain formulatory agents such as suspending, stabilizing and/or dispersing agents.

For example, a parenteral preparation may be a sterile injectable solution or suspension in a nontoxic parenterally acceptable diluent or solvent (e.g., as a solution in 1,3-butanediol). Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed including synthetic mono- or diglycerides. In addition, fatty acids such as oleic acid may be used in the parenteral preparation.

Alternatively, the composition may be in powder form for constitution with a suitable vehicle, such as sterile pyrogen-free water, before use. For example, a composition suitable for parenteral administration may comprise a sterile isotonic saline solution containing between 0.1 percent and 90 percent weight per volume of the composition or composition combination. By way of example, a solution may contain from about 5 percent to about 20 percent, more preferably from about 5 percent to about 17 percent, more preferably from about 8 to about 14 percent, and still more preferably about 10 percent of the composition. The solution or powder preparation may also include a solubilizing agent and a local anesthetic such as lignocaine to ease pain at the site of the injection. Other methods of parenteral delivery of compositions will be known to the skilled artisan and are within the scope of the invention.

### Other Systems of Administration

Various other delivery systems are known in the art and can be used to administer the compositions of the invention. Moreover, these and other delivery systems may be combined and/or modified to optimize the administration of the compositions of the present invention. In some embodiments, the formulation can be aerosolized.

### Administration by Inhalation

Pulmonary delivery of therapeutic agents can offer several advantages over other modes of delivery. These advantages include rapid onset, the convenience of patient self-administration, the potential for reduced drug side-effects, ease of delivery by inhalation and the elimination of needles. Inhalation therapy is capable of providing a drug delivery system that is easy to use in an inpatient or outpatient setting, results in very rapid onset of drug action, and produces minimal side effects. An inhaler is a medical device for delivering a drug to the lungs of a patient. Inhalers or inhalation devices that deliver their content in the forms of liquid mists and powder in aerosol forms, are in common use today.

The present invention is suitable for use in treating human respiratory disease caused by RNA viruses, or any other virus. It is not a goal of this invention to induce protein expression by the RNA delivered, although it is possible that by some unknown method a very small percentage of the RNA delivered enters the cytoplasm of the cell. For example, if a patient has flu-symptoms and the invention of the present disclosure is used and the patient inhales the prepared RNA, since the RNA is not specifically prepared to be delivered into the cell, it is unlikely for the RNA to be expressed into protein in any significant amount. Also, in this example, since our RNA is not selected for a certain protein to assist in this case of respiratory illness, even if an insignificant amount of RNA enters the cell and translation of the RNA into protein occurs, again unlikely to alter the outcome of the illness. In this paragraph, the factor described is RNA but any of the PRR agonists and/or adjuvants described can also replace RNA in the discussion of this paragraph.

Appropriate dosage forms for such administration may be prepared by conventional techniques. In preferred embodiments the methods involve administering to the subject a factor via inhalation of an aerosolized dose. Appropriate dosage forms for such administration, such as an aerosol formulation or a metered dose inhaler, may be prepared by conventional techniques. It will also be appreciated by one of skill in the art that the optimal course of treatment can be ascertained by those skilled in the art using conventional course of treatment determination tests. Generally, the efficacy of administering any of these compositions is adjusted by measuring any change in the immune response or following other clinical parameters.

In other embodiments, cells are scraped from the mucosal surface of a patient's mouth or other easily accessible mucosal surface and the cells are prepared for inhalation into the respiratory tract. During the death of these cells, various types of DAMPs are released. Adjusting the timing of the death of these cells can affect the types and amounts of DAMPs released. From the time the cells are harvested to up to hours later, cells and all of the factors released (all derived from the harvested cell) can be administered as an inhaled therapeutic. There will be varying concentrations of DAMPs present in the therapeutic inhaled solution based on the amount of time elapse from harvesting from patient's mucosa, based on the technique used to harvest the cells and lastly based on the method of cell destruction.

Yet further, another object of the invention and a huge advantage of the invention of this disclosure is to provide treatment to almost any respiratory RNA virus that affects humans and animals. A significant advantage is that if a new strain of influenza virus or coronavirus begins to infect humans (jump from animal to human is a rare but dangerous event), there is much less ramp up time to having a formulation ready for an initial human trial unlike most medications and there is not a minimum 8-month period for production of a vaccine. The preparation of most medications require toxicology assays and studies to determine side effect profiles.

In yet another embodiment of this invention, the first dose (of factor or factors in combination with a therapeutic to decrease white blood cell migration and/or activation) is followed by a second dose at a later time (as early as 6 hours after the first dose and up to 48 hours after the first dose) and the second dose may be a factor or factors with or without addition of the therapeutic to limit white blood cell migration and/or activation. The dose of factor may be slightly less, equal, or slightly greater than the first dose. A third dose may also be added at a later time (as early as 6 hours after the second dose and up to 48 hours after the second dose) and the third dose may be a factor or factors with or without addition of the therapeutic to limit white blood cell migration and/or activation. The dose of factor may be slightly less, equal, or slightly greater than the second dose. A course of treatment may consist of a single administration or of multiple administrations. A multiple dose regime may consist of two, three, four, or up to five administrations. Doses may be spaced by a time interval of 4 hours, up to 8 hours, up to 12 hours, up to 24 hours, or up to 2 days. For chronic virus infections, subjects may undergo many courses of treatment.

An “effective dose” refers to amounts of the factor or factors that are sufficient to affect the course and the severity of the disease, leading to quicker resolution of symptoms. Administration of the factor or steroid may be in a variety of ways, e.g. by intravenous, intramuscular, oral (option for steroid) or aerosolized. Formulations for respiratory administration are preferred for the composition of factor or factors. The “effective dose” useful for treating these viral diseases may be determined using methods known to one skilled in the art. As is well known in the medical profession, dosages for any given patient depends on many factors including patient's size, age, sex, the particular factors to be administered and route of administration.

### Active Ingredient Kits

In various embodiments, the present invention can also involve kits. Such kits can include the compositions of the present invention and, in certain embodiments, instructions for administration. When supplied as a kit, the different components of the composition can be packaged in separate containers and admixed immediately before use. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components. In addition, if more than one route of administration is intended or more than one schedule for administration is intended, the different components can be packaged separately and not mixed prior to use. In various embodiments, the different components can be packaged in one composition for administration together.

Kits may also include reagents in separate containers such as, for example, sterile water or saline to be added to a lyophilized active component packaged separately. For example, sealed glass ampules may contain lyophilized phosphatases and in a separate ampule, sterile water, sterile saline or sterile each of which has been packaged under a neutral non-reacting gas, such as nitrogen. Ampules may consist of any suitable material, such as glass, organic polymers, such as polycarbonate, polystyrene, ceramic, metal or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied as an electronic-readable medium, such as a thumb drive, CD-ROM, DVD-ROM, video, audio, and the like. Detailed instructions may not be physically associated with the kit; instead, a user may be directed to an Internet web site specified by the manufacturer or distributor of the kit.

In the invention of this disclosure, the factor is administered to the patient to more quickly signal a greater number of cells to reduce protein production and prepare an “anti-viral state”. At a very low dose of a given factor, there will be minimal increase in white blood cell movement and white blood cell release of destructive enzymes. At a higher dose of a given factor, the signaling is more quickly achieved but with greater risk of recruitment of white blood cells and their migration to the affected area and significantly greater white blood cell release of destructive enzymes. In this situation, administering a corticosteroid or similar therapeutic will be necessary to control the damage induced by the increased activity of the white blood cells. The dose at which this will become necessary will be different for most factors. Also, the method of administration of the respective factor will affect the need for corticosteroid of similar therapeutic. For example, administration of the factor intramuscularly will still result in local activation of PRRs that will result in production of interferon and these interferons will travel throughout the body including the lung. But because of the blood lung barrier, much less interferon will be present in the alveolar epithelial area space and so there will be less need for a systemic corticosteroid or similar therapeutic. Intramuscular injection of steroid will create more adverse patient symptoms such as muscle-ache and fever and less effectivity in signaling the lung alveolar epithelial cells. However, if a corticosteroid is contraindicated in a patient, this may be the better option for factor administration.

Accordingly, an object of the invention of this disclosure is to provide a method of treating a viral respiratory tract infection or one or more related symptoms thereof comprising administering to a subject a combination of a therapeutically effective amount of a PRR agonist and a therapeutically effective amount of a medication to decrease white blood cell migration.

Another object of the present invention is to provide methods and compositions for the treatment of patients infected with many other viruses. Non-limiting examples of viruses include the herpes virus (e.g., human cytomegalomous virus (HCMV), herpes simplex virus 1 (HSV-1), herpes simplex virus 2 (HSV-2), varicella zoster virus (VZV), Epstein-Barr virus), influenza A virus and Hepatitis C virus (HCV) or a picornavirus such as Coxsackievirus B3 (CVB3). Other viruses may include, but are not limited to, the hepatitis B virus, HIV, poxvirus, hepadavirus, retrovirus, and RNA viruses such as flavivirus, togavirus, coronavirus, Hepatitis D virus, orthomyxovirus, paramyxovirus, rhabdovirus, bunyavirus, filo virus, Adenovirus, Human herpesvirus, type 8, Human papillomavirus, BK virus, JC virus, Smallpox, Hepatitis B virus, Human bocavirus, Parvovirus B19, Human astrovirus, Norwalk virus, coxsackievirus, hepatitis A virus, poliovirus, rhinovirus, Severe acute respiratory syndrome virus, Hepatitis C virus, yellow fever virus, dengue virus, West Nile virus, Rubella virus, Hepatitis E virus, and Human immunodeficiency virus (HIV). In some cases, the virus is an enveloped virus. Examples include, but are not limited to, viruses that are members of the hepadnavirus family, herpesvirus family, iridovirus family, poxvirus family, flavivirus family, togavirus family, retrovirus family, coronavirus family, filovirus family, rhabdovirus family, bunyavirus family, orthomyxovirus family, paramyxovirus family, and arenavirus family. Other examples include, but are not limited to, Hepadnavirus hepatitis B virus (HBV), woodchuck hepatitis virus, ground squirrel (Hepadnaviridae) hepatitis virus, duck hepatitis B virus, heron hepatitis B virus, Herpesvirus herpes simplex virus (HSV) types 1 and 2, varicella-zoster virus, cytomegalovirus (CMV), human cytomegalovirus (HCMV), mouse cytomegalovirus (MCMV), guinea pig cytomegalovirus (GPCMV), Epstein-Barr virus (EBV), human herpes virus 6 (HHV variants A and B), human herpes virus 7 (HHV-7), human herpes virus 8 (HHV-8), Kaposi's sarcoma-associated herpes virus (KSHV), B virus Poxvirus vaccinia virus, variola virus, smallpox virus, monkeypox virus, cowpox virus, camelpox virus, ectromelia virus, mousepox virus, rabbitpox viruses, raccoonpox viruses, molluscum contagiosum virus, orf virus, milker's nodes virus, bovin papullar stomatitis virus, sheeppox virus, goatpox virus, lumpy skin disease virus, fowlpox virus, canarypox virus, pigeonpox virus, sparrowpox virus, myxoma virus, hare fibroma virus, rabbit fibroma virus, squirrel fibroma viruses, swinepox virus, tanapox virus, Yabapox virus, Flavivirus dengue virus, hepatitis C virus (HCV), GB hepatitis viruses (GBV-A, GBV-B and GBV-C), West Nile virus, yellow fever virus, St. Louis encephalitis virus, Japanese encephalitis virus, Powassan virus, tick-borne encephalitis virus, Kyasanur Forest disease virus, Togavirus, Venezuelan equine encephalitis (VEE) virus, chikungunya virus, Ross River virus, Mayaro virus, Sindbis virus, rubella virus, Retrovirus human immunodeficiency virus (HIV) types 1 and 2, human T cell leukemia virus (HTLV) types 1, 2, and 5, mouse mammary tumor virus (MMTV), Rous sarcoma virus (RSV), lentiviruses, Coronavirus, severe acute respiratory syndrome (SARS) virus, Filovirus Ebola virus, Marburg virus, Metapneumoviruses (MPV) such as human metapneumovirus (HMPV), Rhabdovirus rabies virus, vesicular stomatitis virus, Bunyavirus, Crimean-Congo hemorrhagic fever virus, Rift Valley fever virus, La Crosse virus, Hantaan virus, Orthomyxovirus, influenza virus (types A, B, and C), Paramyxovirus, parainfluenza virus (PIV types 1, 2 and 3), respiratory syncytial virus (types A and B), measles virus, mumps virus, Arenavirus, lymphocytic choriomeningitis virus, Junin virus, Machupo virus, Guanarito virus, Lassa virus, Ampari virus, Flexal virus, Ippy virus, Mobala virus, Mopeia virus, Latino virus, Parana virus, Pichinde virus, Punta toro virus (PTV), Tacaribe virus and Tamiami virus. In some embodiments, the virus is a non-enveloped virus, examples of which include, but are not limited to, viruses that are members of the parvovirus family, circovirus family, polyoma virus family, papillomavirus family, adenovirus family, iridovirus family, reovirus family, birnavirus family, calicivirus family, and picornavirus family. Specific examples include, but are not limited to, canine parvovirus, parvovirus B19, porcine circovirus type 1 and 2, BFDV (Beak and Feather Disease virus, chicken anaemia virus, Polyomavirus, simian virus 40 (SV40), JC virus, BK virus, Budgerigar fledgling disease virus, human papillomavirus, bovine papillomavirus (BPV) type 1, cotton tail rabbit papillomavirus, human adenovirus (HAdV-A, HAdV-B, HAdV-C, HAdV-D, HAdV-E, and HAdV-F), fowl adenovirus A, bovine adenovirus D, frog adenovirus, Reovirus, human orbivirus, human coltivirus, mammalian orthoreovirus, bluetongue virus, rotavirus A, rotaviruses (groups B to G), Colorado tick fever virus, aquareovirus A, cypovirus 1, Fiji disease virus, rice dwarf virus, rice ragged stunt virus, idnoreovirus 1, mycoreovirus 1, Birnavirus, bursal disease virus, pancreatic necrosis virus, Calicivirus, swine vesicular exanthema virus, rabbit hemorrhagic disease virus, Norwalk virus, Sapporo virus, Picornavirus, human polioviruses (1-3), human coxsackieviruses A1-22, 24 (CA1-22 and CA24, CA23 (echovirus 9)), human coxsackieviruses (B1-6 (CB1-6)), human echoviruses 1-7, 9, 11-27, 29-33, vilyuish virus, simian enteroviruses 1-18 (SEV1-18), porcine enteroviruses 1-11 (PEV1-11), bovine enteroviruses 1-2 (BEV1-2), hepatitis A virus, rhinoviruses, hepatoviruses, cardio viruses, aphthoviruses and echoviruses.

### Calculating Dosing

The dosage determination for the components of the composition depend on the actual component and or components used. The components or factors that accomplish each of the fundamental strategies are different and dosages for each of the components/factors used from each of the fundamental strategies can be determined using any of the methods described below. Then, later, in an in vivo trial, the composition (including components that activate parts of the immune system and components that inhibit other parts of the immune system) can also be evaluated using the methods described herein to help determine an effective combination dose. As an example, the dosage determination for any of the PRR agonists selected for the composition will be much lower than the dosages for the white blood cell migration/activation inhibitor. The dosage determination for white blood cell migration/activation inhibition may be determined using standard techniques, by a pharmacologist of ordinary skill in the art. Much of the discussion below describing particular dosage determinations and ranges will be directed towards determining the correct dosage for PRR agonists but the general concepts are applicable to dosage calculations for factors from each of the fundamental strategies. As the dosage for the PRR agonist within the composition is increased, it will be clear that the dosage of the white blood cell activation inhibitor (component that inhibits parts of the immune system) will typically be increased.

Precise formulations and dosages will depend on the exact PRR agonist and the exact white blood cell migration inhibitor (e.g. corticosteroid) used in the composition and may be determined using standard techniques, by a pharmacologist of ordinary skill in the art. In addition, in vivo and/or in vitro assays may also be employed to provide improved optimal dosage ranges since, as an example, a higher PRR agonist dosage will require a higher white blood cell migration inhibitor dosage. The precise dose to be used in the composition will also depend on the route of administration. Predicted suitable doses of the PRR agonist component of the composition include, but are not limited to, 1 ng/kg to 2 mg/kg per day. Effective dosage may be extrapolated from dose response curves derived from in vitro or animal model test systems. The length of treatment will generally be 2 to 4 days but may be up to two to four weeks, depending on the specific virus. Methods of preparing parenterally, orally, or intranasally compositions are well known in the art and described in detail in various sources as described below. Any discussion in dosage calculation derivation regarding a PRR agonist also applies to factors from any of the other fundamental strategies described in this specification.

The specific safe and effective amount of PRR agonist to be used in the composition will vary with factors as described above, including the specific route of administration, the carrier employed, and the desired dosage regimen. For example, in various embodiments, the PRR agonist within the composition may be administered to a patient in low doses of less than or about 1 mg/kg, of less than or about 1 μg/kg, of less than or about 300 ng/kg, less than or about 30 ng/kg or less than or about 3 ng/kg.

The dosage of the composition may also consider pharmacokinetic paramaters well known in the art, i.e., rate of absorption of the composition, bioavailability, rate of metabolism of the composition, etc. See, e.g., Johnson (1995), Hidalgo-Aragones (1996), Groning (1996), Brophy (1983), Remington's (2020).

Therapeutically effective amounts of the various factors used in the composition can also be determined in animal studies. The dose can be adjusted to achieve maximal efficacy based on the capabilities of the ordinarily skilled artisan. Patient doses of the PRR agonist as described herein may typically range from about 1 ng/kg to about 2 mg/kg, even more typically from about 30 ng/kg to about 500 μg/kg, and most typically from about 50 ng/kg to 500 ng/kg although daily doses may be more than 1 mg/kg/day or less than 1.0 ng/kg/day. This includes amounts equal to or less than 0.9, 0.8, 0.7, 0.6, 0.5, 0.4, 0.3, 0.2, 0.1 mg/kg per day. Adjustment of established dose ranges are well within the ability of those skilled in the art.

An “effective amount’ of the composition of this invention includes a therapeutically effective amount or a prophylactically effective amount. A “therapeutically effective amount” refers to an amount effective to achieve the desired therapeutic result, such as reduction or elimination of the virus, a quicker recovery rate than expected without the therapeutic composition, and/or a less severe clinical course of the virus than without the treatment of this invention. Routes of administration and dosage ranges described herein are exemplary only and are not meant to limit the route of administration and dosage ranges that may be selected by medical practitioners.

The present technology provides methods for treating or preventing virus infections in a subject comprising administering to the subject a therapeutically effective amount of a composition comprising a factor or factors chosen from at least one of the fundamental strategies described within this specification. The methods and compositions of the present technology comprise the administration of compositions having dosage levels suitable for a safe and effective therapeutic effect in the subject, without undue adverse side effects, consistent with a reasonable risk/benefit ratio. The biological activity of a particular PRR agonist may be strikingly different from the biological activity of another PRR agonist and one skilled in the art would appropriately adjust the dosage of a more biologically active PRR agonist. The compositions can include a pharmaceutically acceptable carrier. Depending on the route of administration, an acceptable carrier may be tailored for the particular route of administration. For example, solid form carriers include powders, tablets, capsules, etc. Carriers can also act as diluents, flavoring agents and binders. Suitable carriers include sugars, lactose, dextrin, methyl cellulose and pectin, but those skilled in the art know that the list is extensive.

The amount of PRR agonist used in the compositions and methods of the present invention is an amount which produces an effective increase in the immune response in the host, especially the portions of the immune response that decrease protein production within cells and/or increase ribonuclease activity within cells and/or increases production of factors that signal other cells to decrease protein production and/or increase ribonuclease activity. There are many quantitative measurements that can be performed to assess the effective increase in in the immune response (decreasing protein production and/or increased ribonuclease activity). Increased immune system activity typically leads to decreased protein production and/or increased ribonuclease activity. So, many markers that quantify immune or inflammatory activity are also correlated with decreased protein production and/or increased ribonuclease activity and the quantitative measurement (including but not limited to mass/concentration/biological activity) of many of these biological activities may be used to quantify increased immune system activity. Methods to measure immune system activity are well known to those skilled in the art but some examples of assays include quantification of cytokines, interferon (including IFN-α, IFN-β, IFN-γ) and TNF. There are thousands of pro-inflammatory genes whose products can be assayed. The goal of this invention is to increase the activity of the portion of the immune system response that decreases protein production and/or increases ribonuclease activity, and/or increases production of signaling factors that accomplish the fundamental strategies discussed herein. Additional illustrative cytokines that may be assayed to assess the biological effects of the PRR agonist include but are not limited to IL-la, IL-la, IL-2, IL-3, IL-4, IL-5, IL-6, IL-7, IL-10, IL-12, IL-15, IL-18, IL-23 TGF-β, MIP-1α, Mix-1β, MCP-1, GM-CSF and G-CSF.

Decreasing white blood cell migration and/or activation is one of the fundamental strategies described within this specification. There are many assays and techniques/methods available that can demonstrate the effectiveness of a white blood cell migration/activation inhibitor. As an example, PMN (polymorphonuclear) cell migration inhibition can be shown using purified human granulocyte cells from peripheral blood, re-suspending in migration buffer, using a transwell filter system and a control chemokine (such as IL-8, a neutrophil chemoattractant) and adding increasing amounts of a white blood cell migration inhibitor such as corticosteroids in increasing amounts in separate chambers, and then counting the neutrophils that have migrated to the lower chamber of the transwell system. Higher doses of corticosteroids decrease the migration of neutrophils across this transwell system in response to a controlled amount of chemoattractant.

The dosage range for a corticosteroid ranges from about 0.01 μg to about 100 mg per day. In another embodiment, the dosage for a corticosteroid ranges from about 1.0 μg to about 50 μg per day, or about 10 μg to about 100 μg per day, or about 100 μg to about 500 μg per day, or about 500 μg to about 1000 μg per day, or about 1.0 mg per day to about 10 mg per day, or about 5 mg per day to about 100 mg per day.

Preferred embodiments are described in the following example. Other embodiments within the scope of the claims herein will be apparent to one skilled in the art from consideration of the specification or practice of the invention as disclosed herein. It is intended that the specification, together with the examples, be considered exemplary only, with the scope and spirit of the invention being indicated by the claims, which follow the example.

### Example

### Materials and Methods: Cell Culture A549 Epithelial Cells

Cellular experimentation was carried out using carcinoma human lung cell line A549 (Sigma catalog no. 86012804). Initial cell line was prepared and thawed per ATCC A549 product sheet. Immediately upon arrival, cells were removed from dry ice packaging and placed in a prewarmed water bath at 37 C. Cells were thawed<1 minute until minute amount of ice was in suspension. Cell cryovial was swirled to evenly distribute cells and progress thawing. The cells were aseptically transferred to the biological safety cabinet and wiped with 70% ethanol solution prior to opening. Cells were transferred dropwise into a centrifuge tube containing prewarmed culture media. After transfer, the cells were centrifuged, supernatant aseptically decanted, and resuspended in culture media. Cultures seeded in Nunc Cell Culture Treated T25 EasYFlasks with filtered caps (ThermoFisher catalog no. 156367) for initial growth before expansion and separation into subcultures.

Cells were incubated at 37 C and 5% CO2 with >90% humidity within the cell incubator. The A549 cell line was cultured in Nutrient Mixture F12 Ham Kaighn's Modified (F12K) cell culture media (Sigma catalog no. N3520) with 10% Fetal Bovine Serum (Sigma catalog no. F2442) per Sigma Aldrich F12K product data sheet and preparation instructions. Cell subcultures were grown in Nunc Cell Culture Treated T75 EasYFlasks with filtered caps (ThermoFisher catalog no. 156499).

Subcultures were passaged within a 48-hour time period from the initial culture seeding. Passaging was carried out when cultures reached 50-60% confluency. Culture media was aspirated off and discarded. Cells were then washed using StableCell Trypsin Solution (Sigma catalog no. T2601). Following initial washing, trypsin solution was added to culture for cell layer dispersion. Once dispersed, cells were transferred to a centrifuge tube, centrifuged, resuspended in fresh sterile culture media and seeded at predetermined seeding density.

Within 24 hours after seeding, culture media was aspirated and discarded. Subcultures were washed with 1× Dulbecco's Phosphate Buffered Saline w/o CA++ & Mg++ (Sigma catalog no. BSS-1006-B) every 24 hrs and culture media was renewed.

Preparation of Lipopolysaccharides (LPS) and Hydrocortisone: Lipopolysaccharides (LPS) from Escherichia coli 0111:B4 lyophilized powder, y-irradiated (Sigma catalog no. L439) was used for experimentation. LPS was stored, handled and reconstituted per Sigma Aldrich product sheet. LPS pressurized vial was opened under a biological safety cabinet in a sterile environment using BSL-2 safety protocols and standards in place. Once opened, lyophilized powder fragmented into smaller pieces. These smaller components were transferred into silane treated 2 mL amber vials (Sigma catalog no. 27238) using wax weight paper as a funnel.

Once all LPS powder was transferred, sterile Hanks' Balanced Salt solution (Sigma catalog no. H6648) was used for reconstitution. The vial was then vigorously vortexed for 5 minutes to ensure LPS was properly dissolved and mixed in solution. LPS solution was then transferred from vial at predetermined volume using low binding micropipette tips.

Hydrocortisone (Cortisol) (Sigma catalog no. H0888) was used for experimentation. Hydrocortisone was reconstituted to prepare a stock solution for cell culture. A concentration of 50 μg/mL stock solution was created per Sigma Aldrich product information sheet. 1.0 mL of sterile ethanol was added to 1.0 mg of hydrocortisone powder. Solution was gently vortexed for 2 minutes and checked to ensure hydrocortisone was dissolved in solution. 19 mL of sterile Nutrient Mixture F12 Ham Kaighn's Modified (F12K) cell culture media was added to the solution. Final stock solution was prepared, aliquoted and stored for future usage and experimentation.

Experimental Procedure and Image Analysis: A549 cell subcultures were seeded for experimentation. Multiple control and experimental subcultures were created for each trial. Subcultures were grown in Nunc Cell Culture Treated T25 EasYFlasks with filtered caps. Reagents, LPS and Hydrocortisone, were added to subcultures 8 hours after initial seeding when cells had successfully adhered to flasks and were approximately 10-20% confluent. Predetermined and pharmacological levels of LPS and hydrocortisone were added to the experimental subcultures. Images of cell growth were taken at time points of 12, 24, 48, 96 hours; time point zero represents when the reagents were added to the cultures. Cell images were taken using the Amscope Trinocular Inverted Tissue Culture Microscope 40×-640× at 4× objective magnification.

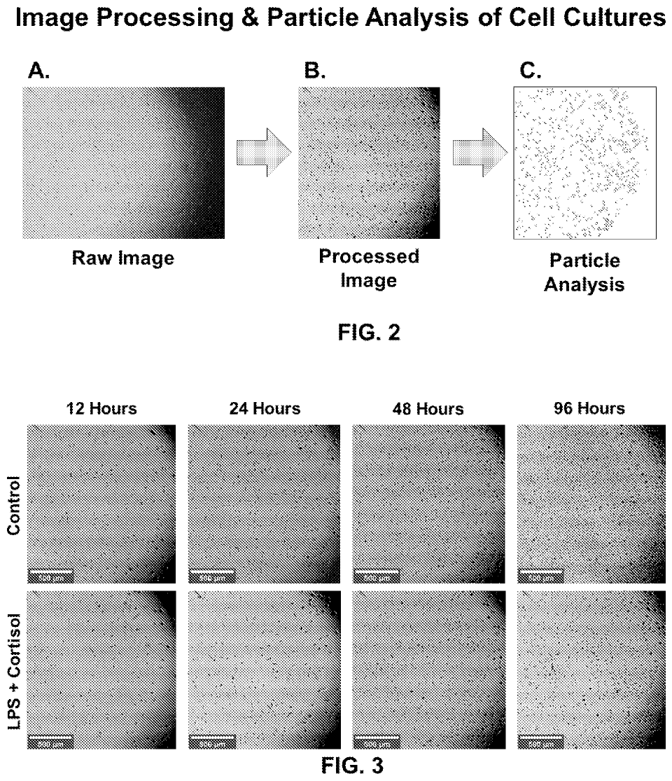

Upon completion of cell culture experimentation, all images and data were recorded and saved for further analysis. ImageJ was used to edit, modify and analyse cell culture images and data. All images were converted to 8 bit black and white, cropped and adjusted contrast, brightness for better visualization and processing of cells. FIG. 2 shows the visual process of how the cellular images were prepared for particle analysis as well as representing the analysis results.

Scale bars were set using a Microscope Stage Calibration Slide for USB Camera 0.01 mm to convert pixel units to micrometers. Scale bars were edited and set to 500 μm for clarity and cell size referencing. Upon completion of image editing, all images were analyzed using ImageJ automated particle counting and analysis algorithm. Images were processed into binary, then converted to mask. ImageJ “analyze particles” program was run to obtain cell counts in all experimental images, shown in FIG. 2.

### Results

Preliminary data shows the population growth differences between a control sample and experimental sample of A549 epithelial cells that have been cultured with LPS and cortisol over time. FIG. 3 qualitatively shows the growth of each cell culture over time. As seen, the control group had a greater density from 24 hours to 96 hours. The initial population density was comparable to the experimental group. It is clear, by 96 hours the control group had expanded exponentially, while the experimental group had approximately doubled from the initial population size.

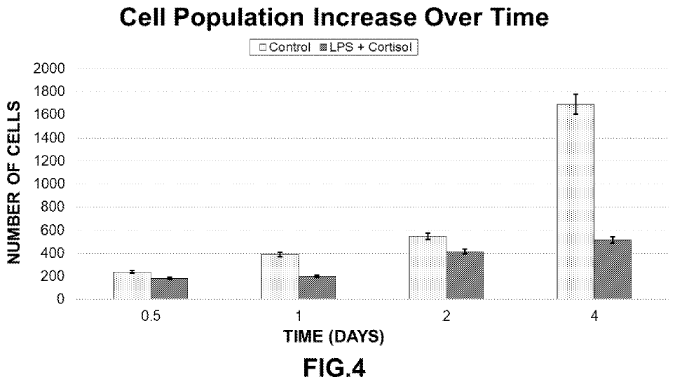

Further cell growth analysis was completed and population data has been represented graphically. FIG. 4 shows cell population increase over time as a bar graph. As shown by FIG. 4, the control group population had increased greatly, with exponential growth between day 2 and day 4, with an overall greater population size at every time point. The experimental group had similar growth patterns up to 24 hrs, but with minimal expansion thereafter. Further, percent change of both populations were calculated with the following equation, where Vfinal is the final count of cells at 96 hours and Vinitial is the initial cell count at 12 hours:

% Population Change=(Vfinal−Vinitial)/(Vinitial)*100

The control population had a percentage change of 607%, while the experimental culture percentage change was 184%. This shows that both populations were increasing and positively expanding in cell density. The control group had a population increase ˜3.3 times greater than the experimental population. This highlights that the control cells grew exponentially more over the entire time period versus the experimental culture.

References
Patents and patent applications

| | |
|---|---|
| CA 2460779 A1; WO 02/074939 A1 | U.S. Pat. No. 007,164,008B2 |
| CA 2669763 C; WO 2008/058766 A1 | U.S. Pat. No. 007,329,409B2 |
| CA 2679843 A1; WO 2008/109669 A1 | U.S. Pat. No. 007,618,641B2 |
| CA 2781314 A1; WO 2011/0945498 A3 | U.S. Pat. No. 007,622,128B2 |
| CA 2899787 A1; WO 2014/118305 A1 | U.S. Pat. No. 007,968,122B2 |
| CN 102988956 A | U.S. Pat. No. 008,153,592B2 |
| CN 103327991 A | U.S. Pat. No. 008,163,281B2 |
| EP 1870468 A2 | U.S. Pat. No. 008,216,567B2 |
| EP 2050455 A1 | U.S. Pat. No. 008,530,515B2 |
| EP 2139501 B1 | U.S. Pat. No. 008,636,979B2 |
| EP 2605791 B1 | U.S. Pat. No. 008,637,013B2 |
| EP 2829281 A1 | U.S. Pat. No. 008,796,243B2 |
| JP 6084655 B2 | U.S. Pat. No. 008,821,884B2 |
| KR 10-2013-0082139 | U.S. Pat. No. 008,980,944B2 |
| U.S. Pat. No. 005,484,589A | U.S. Pat. No. 009,192,649B2 |
| U.S. Pat. No. 005,484,591A | U.S. Pat. No. 009,422,545B2 |
| U.S. Pat. No. 005,658,937A | US 2004/0115178A1 |
| U.S. Pat. No. 005,804,189A | US 2005/0158276A1 |
| U.S. Pat. No. 005,846,789A | US 2007/0042004A1 |
| U.S. Pat. No. 005,871,951A | US 2009/0143831A1 |
| U.S. Pat. No. 005,985,284A | US 2011/0091484A1 |
| U.S. Pat. No. 006,368,604B1 | US 2011/0142817A1 |
| U.S. Pat. No. 006,596,477B1 | US 2011/0218169A1 |
| U.S. Pat. No. 006,800,613B2 | US 2011/0293658A1 |
| U.S. Pat. No. 6,841,345B1 | US 2013/0142825A1 |
| U.S. Pat. No. 007,012,127B1 | US 2013/0237592A1 |
| U.S. Pat. No. 007,048,953B2 | US 2013/0266640A1 |
| US 2013/0295205A1 | WO 2008/123758A1 |
| US 2014/0199347A1 | WO 2009/004339A2 |
| US 2014/0200261A1 | WO 2010/130896A2 |
| US 2014/0371302A1 | WO 2011/044246A1 |
| US 2015/0017202A1 | WO 2011/161491A1 |
| US 2015/0056217A1 | WO 2012/109203A1 |
| US 2015/0064725A1 | WO 2012/175735A1 |
| US 2015/0086649A1 | WO 2013/088136A1 |
| US 2015/0211039A1 | WO 2013/120073A1 |
| US 2015/0315541A1 | WO 2013/140919A1 |
| US 2016/0108108A1 | WO 2013/148072A1 |
| US 2016/0151453A1 | WO 2013/170735A1 |
| US 2016/0264614A1 | WO 2013/182683A1 |
| WO 90/00061 | WO 2014/016417A1 |
| WO 95/08560 | WO 2014/033097A1 |
| WO 96/23002 | WO 2014/113625A1 |
| WO 98/37919 | WO 2014/152027A1 |
| WO 01/78787A2 | WO 2015/058087A1 |
| WO 02/16440A2 | WO 2015/197706A1 |
| WO 02/094296A1 | WO 2016/046218A1 |
| WO 2006/068663A2 | WO 2016/090404A1 |
| WO 2007/053455A2 | WO 2017/013603A1 |
| WO 2007/112966A1 | WO 2017/021791A1 |
| WO 2008/095679A1 | WO 2017/021792A1 |
| WO 2008/119851A1 | WO 2017/200852A1 |

### Non-Patent Literature

Johnson (1995) J. Pharm. Sci. 84:1144-1146
Hidalgo-Aragones (1996) J. Steroid Biochem. Mol. Biol. 58:610-627
Groning (1996) Pharmazie 51:335-341
Brophy (1983) Eur. J. Clin. Pharmacol. 24:103-108
Remington's Pharmaceutical Sciences (2020) 23rd Edition, Editor, Adoboye Adejare, The Science and Practice of Pharmacy
Chaplin (2010) J Allergy Clin Immunol. DOI: 10.1016/j.jaci.2009.12.980
Giudice (2018) Seminars in Immunology 39. DOI: https://doi.org/10.1016/j.smim.2018.05.001
Hartl (2018) J Innate Immun. DOI: 10.1159/000487057
MacDonald (2000) J Nutr. DOI: 10.1093/jn/130.5.15005
Chen (2020) J Immunol. DOI: 10.4049/jimmunol.2000380
Hiemstra (2015) Eur Respir J. DOI: 10.1183/09031936.00141514
Ilinskaya (2014) Mol Biol. DOI: 10.1134/S0026893314040050
Hu (2015) PNAS. DOI: 10.1073/pnas.1510760112
Agrawal (2013) Gerontology. DOI: 10.1159/000350536
Crosse (2018) J Innate Immun. DOI: 10.1159/000484258
Perry (2005) Cell Res. DOI: 10.1038/sj.cr.7290309
Snyder (2017) J Immun. DOI: 10.1155/2017/9361802
Tan (2018) Ox Med. & Cell. DOI: 10.1155/2018/9719584
Anaya J M (2013) El Rosario University Press. Bookshelf ID: NBK459447
Hotamisligil (2016) Cold Spring Harb Perspect Biol. DOI: 10.1101/cshperspect.a006072
West (2009) Am J Physiol Regul Integr Comp Physiol. DOI: 10.1152/ajpregu.00459.2009
Walsh (2013) Cold Spring Harb Perspect Biol. 2013 Jan. 1; 5(1):a012351. DOI: 10.1101/cshperspect.a012351
Hou (2019) Frontiers in plant science vol. 10 646. DOI:10.3389/fpls.2019.00646
West J B (2007) Eur Respir J. DOI: 10.1183/09031936.00133306
Sapolsky (2000) Endocr Rev. DOI: 10.1210/edrv.21.1.0389
Bercovich-Kinori (2016) eLife 2016; 5:e18311. DOI: 10.7554/eLife.18311
Stern A (2017) Cell. DOI: 10.1016/j.cell.2017.03.013
Mussbacher (2019) Front. Immunol. DOI: 10.3389/fimmu.2019.00085
Sudheimer (2014) Front. Aging Neurosci. DOI: 10.3389/fnagi.2014.00153
Braun (2015) Front. Physiol. DOI: 10.3389/fphys.2015.00012
Marciniak (2004) Genes Dev. DOI: 10.1101/gad.1250704
Simmons (2009) Toxicol Sci. DOI: 10.1093/toxsci/kfp140
Twigg H L (2005) Proc Am Thorac Soc. DOI: 10.1513/pats.200508-089JS
Koczera (2016) Int J Mol Sci. DOI: 10.3390/ijms17081278
Saha S K (2017) Int J Mol Sci. DOI: 10.3390/ijms18071544
Tan B J (2013) Int J Oncol. DOI: 10.3892/ijo.2013.1843
Perales-Linares R (2013) Immunology. DOI: 10.1111/imm.12143
Reynolds H Y (1988) Mayo Clin Proc. DOI: 10.1016/s0025-6196(12)64949-0
Burnett D (1986) Immunoglobulins in the lung. Thorax; 41:337-344.
Garcia M A (2006) Microbiology and Molecular Biology Reviews. DOI: 10.1128NIMBR.00027-06
Oh S J (2019) Immune Netw. DOI: https://doi.org/10.4110/in.2019.19.e37
van 't Wout E F (2014) Am J Respir Cell Mol Biol. DOI: 10.1165/rcmb.2014-0019TR
Pakos-Zebrucka K (2016) EMBO Rep. 17(10):1374-1395. DOI: 10.15252/embr.201642195
Platanias L C (2005) Nat Rev Immunol. DOI: 10.1038/nri1604
Eigenmann M J (2017) J Physiol. DOI: 10.1113/JP274819
Washington University in St. Louis. (2014) ScienceDaily. <www.sciencedaily.com/releases/2014/11/141113122235.htm>.
Lamech (2015) J Cell Biol. DOI: https://doi.org/10.1083/jcb.201503107
Wagner D K (1987) J Clin Microbiol. DOI: 10.1128/JCM.25.3.559-562.1987
Cunningham-Rundles (2009) Mount Sinai School of Medicine. DOI: https://doi.org/10.1111/j.1365-2249.2009.03952.x
Sandra N (2014) J Mol Bio. DOI: https://doi.org/10.1016/j.jmb.2013.11.024
Houseley J (2009) Cell. DOI: 10.1016/j.cell.2009.01.019
Huang J (2008) Mol Cell Biol. DOI: 10.1128/MCB.00289-08
Grover A (2002) Cell Stress Chaperones. DOI: 10.1379/1466-1268(2002)007<0001:mbosr>2.0.co;2
Pardi, N. (2018) Nat Rev Drug Discov 17. DOI: https://doi.org/10.1038/nrd.2017.243
Santoro M G (2003) EMBO J. DOI: 10.1093/emboj/cdg267
Reed S (2013) Nat Med 19. DOI: https://doi.org/10.1038/nm.3409
Borden E C (2007) Nat Rev Drug Discov. DOI: 10.1038/nrd2422
Gameiro P A (2018) Cell Rep. DOI: 10.1016/j.celrep.2018.07.021
Filomeni, G (2015) Cell Death Differ 22. DOI: https://doi.org/10.1038/cdd.2014.150
Sahin, U (2017) Nature 547. DOI: https://doi.org/10.1038/nature23003
Shenton D (2006) J Biol Chem. DOI: 10.1074/jbc.M601545200
Takeuchi O (2010) Cell. DOI: 10.1016/j.cell.2010.01.022
Rojas M (2010) J Virol. DOI: 10.1128/JVI.00625-10
Schulz O (2010) Cell Host Microbe. DOI: 10.1016/j.chom.2010.04.007
Kim K J (2003) Am J Physiol Lung Cell Mol Physiol. DOI: 10.1152/ajplung.00235.2002
Deb A (2001) J Immunol. doi: 10.4049/jimmunol.166.10.6170
MATER METHODS 2012; 2:201. Labome. DOI: //dx.doi.org/10.13070/mm.en.2.201
Manivannan P (2020) J Virol. DOI: 10.1128/JVI.00205-20.
Onomoto, K (2021) Cell Mol Immunol 18. DOI: https://doi.org/10.1038/s41423-020-00602-7
Topf U (2018) Nat Commun. DOI: 10.1038/s41467-017-02694-8
Zainol, M. I. B. (2019) Sci Rep 9. DOI: https://doi.org/10.1038/s41598-019-56914-w
White, E. Z. (2020) Sci Rep 10. DOI: https://doi.org/10.1038/s41598-020-68668-x
Yano, Ken-ichi (2017) IntechOpen. DOI: 10.5772/intechopen.69782
Karikó K (2005) Immunity. DOI: 10.1016/j.immuni.2005.06.008
Lin S (2005) Cell Microbiol. DOI: 10.1111/j.1462-5822.2005.00593.x
Xia Z (2017) Front Physiol. doi: 10.3389/fphys.2017.00434
Land W G (2015) Sultan Qaboos Univ Med J. 15(2):e157-70. Epub 2015 May 28. PMID: 26052447
Kawasaki T (2014) Front Immunol. DOI: 10.3389/fimmu.2014.00461

In view of the above, it will be seen that several objectives of the invention are achieved and other advantages attained.

As various changes could be made in the above methods and compositions without departing from the scope of the invention, it is intended that all matter contained in the above description and shown in the accompanying drawings shall be interpreted as illustrative and not in a limiting sense.

All references cited in this specification, including but not limited to patent publications and non-patent literature, and references cited therein, are hereby incorporated by reference. The discussion of the references herein is intended merely to summarize the assertions made by the authors and no admission is made that any reference constitutes prior art. Applicants reserve the right to challenge the accuracy and pertinence of the cited references.

As used herein, in particular embodiments, the terms “about” or “approximately” when preceding a numerical value indicates the value plus or minus a range of 10%. Where a range of values is provided, it is understood that each intervening value, to the tenth of the unit of the lower limit unless the context clearly dictates otherwise, between the upper and lower limit of that range and any other stated or intervening value in that stated range is encompassed within the disclosure. That the upper and lower limits of these smaller ranges can independently be included in the smaller ranges is also encompassed within the disclosure, subject to any specifically excluded limit in the stated range. Where the stated range includes one or both of the limits, ranges excluding either or both of those included limits are also included in the disclosure.

The indefinite articles “a” and “an,” as used herein in the specification and in the embodiments, unless clearly indicated to the contrary, should be understood to mean “at least one.”

The phrase “and/or,” as used herein in the specification and in the embodiments, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements can optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the embodiments, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the embodiments, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of” “Consisting essentially of,” when used in the embodiments, shall have its ordinary meaning as used in the field of patent law.

As used herein in the specification and in the embodiments, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements can optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

What is claimed is:

1. A method of treating a mammal for a virus infection, the method comprising
(a) increasing ribonuclease amount or activity in a cell infected by the virus;
(b) decreasing protein production in a cell infected by the virus;
(c) activating an innate immune system in the mammal;
(d) reducing migration and/or activation of white blood cells in the mammal;
or any combination thereof.

2. The method of claim 1, wherein ribonuclease amount or activity in the cell is increased through cell signaling.

3. The method of claim 1, wherein ribonuclease amount or activity in the cell is increased by inducing ribonuclease production and/or increasing oxidative stress in the cell to oxidize ribonuclease inhibitor (RI).

4. (canceled)

5. The method of claim 3, wherein ribonuclease production is induced by administering interferon and/or stimulating interferon production.

6. The method of claim 5, wherein interferon production is stimulated by activating the innate immune system and/or treating the cell with bacterial lipopolysaccharide (LPS) and/or an adjuvant and/or a PRR agonist.

7. The method of claim 1, wherein protein production in the cell is decreased by increasing oxidative stress in the cell.

8-10. (canceled)

11. The method of claim 1, wherein the innate immune system in the mammal is activated by treatment of the mammal with an adjuvant and/or a PRR agonist.

12. The method of claim 1, wherein migration and/or activation of white blood cells is reduced in the mammal by administering a glucocorticoid, colchicine, cyclosporine, and/or a non-steroidal anti-inflammatory medication to the mammal.

13. The method of claim 1, wherein at least two of (a), (b), (c), (d) are executed.

14-15. (canceled)

16. The method of claim 1, wherein the mammal is a human.

17. The method of claim 1, wherein the virus is an RNA virus.

18. The method of claim 1, wherein the virus is a DNA virus.

19. The method of claim 1, wherein the virus is a respiratory virus.

20. The method of claim 19, wherein the virus is SARS-CoV-2.

21-22. (canceled)

23. The method of claim 1, wherein the treatment is executed within three days of the initial virus infection.

24. The method of claim 1, wherein the treatment is executed within one day of the initial virus infection.

25. A composition comprising (a) a compound (I) that increases ribonuclease amount or activity in a cell in a mammal infected by a virus, (b) a compound (II) that decreases protein production in a cell in a mammal infected by a virus, (c) a compound (III) that activates an innate immune system in the mammal, (d) a compound (IV) that reduces migration and/or activation of white blood cells in the mammal, or any combination thereof,
wherein the composition is effective in treating a virus infection when administered to a mammal having the virus infection.

26-31. (canceled)

32. The composition of claim 25, wherein compound (I), compound (II), compound (III) and/or compound (IV) is selected from the group consisting of interferon, a stimulant of interferon production, LPS, an adjuvant, a PRR agonist, a glucocorticoid, colchicine, cyclosporine, a non-steroidal anti-inflammatory medication, a CLR agonist, a NOD-like receptor agonist, A RIG-I-like receptor agonist, a DAMP molecule, a PAMP molecule and any combinations thereof.

33. The composition of claim 25, wherein the composition is effective in treating the virus infection when administered to a mammal within three days of the initial virus infection.

34. The composition of claim 25, wherein the composition is effective in treating the virus infection when administered to a mammal within one day of the initial virus infection.

35-40. (canceled)
